(19) United States
(12) Patent Application Publication
(10) Pub. No.: US 2007/0154962 A1
(43) Pub. Date: Jul. 5, 2007

(54) Bcl-2 promoted cell death

(76) Inventors: Giulio Taglialatela, Dickinson, TX (US); Bryce Portier, Hitchcock, TX (US)

Correspondence Address: GORDON & REES LLP, 101 WEST BROADWAY, SUITE 1600, SAN DIEGO, CA, 92101, US

(21) Appl. No.: 11/317,725

(22) Filed: Dec. 23, 2005

(51) Int. Cl. G01N 33/574

(52) U.S. Cl. 435/7.23

(57) ABSTRACT

The invention is directed towards a method of screening compounds that disrupt Bcl-2/FKBP38 binding and thereby induce apoptosis. The invention is also directed towards a method of promoting apoptotic cell death in Bcl-2 producing cells or tissues by contacting said cells or tissues with a sufficient amount of BH4 peptide or mimetic thereof to inhibit binding of Bcl-2 and FKBP38. Additionally, the invention is directed towards a method of purging malignant, Bcl-2 producing cells from a mixed population of cells, by contacting the mixed population with a sufficient amount of BH4 peptide or a mimetic thereof to disrupt Bcl-2/KBP38 binding and trigger apoptosis in Bcl-2 producing cells. The mixed population of cells can be in or from an individual.

### CROSS-REFERENCE TO RELATED APPLICATIONS

This application is based on, and claims the benefit of, U.S. Provisional Application No. 60/639,081, filed Dec. 22, 2004, entitled Bcl-2 PROMOTED CELL DEATH, and is incorporated herein by reference.

### FIELD OF THE INVENTION

The present invention relates to the fields of oncology, genetics and molecular biology. More particular the invention relates to the a method for screening compounds that influence the Bcl-2 BH4-domain mediated binding between Bcl-2 and FKBP38; a method of promoting apoptotic cell death using the identified compound; and a method of purging malignant cells from a mixed population of cells using the identified compound.

### BACKGROUND OF THE INVENTION

Apoptosis plays a fundamental role in the maintenance of tissue function and structural integrity by eliminating unwanted, unnecessary or damaged cells (Chao D T, et al., (1998); Bossy-Wetzel E and Green DR, (1999); Kroemer G and Reed J C (2000); Cory S and Adams J M (2002). Failure of the apoptotic process is an important component of many human cancers as the inability of cells to undergo physiologically programmed apoptotic cell death is an inherent characteristic of their malignant transformation (Konopleva M, et al., (1999); Fisher D E (2001); Konstantinidou A E, et al., (2002)). Apoptosis resistance is also critically involved in the development of chemotherapy drug resistance, one of the main causes of cancer therapy failure (Reed J C (1995); Juin P, et al., (2004); Pommier Y, et al., (2004)).

Proteins of the Bcl-2 family are the best characterized effectors and modulators of cell apoptosis (Allen R T, et al., (1998); Bruckheimer E M, et al., (1998); Chao and Korsmeyer, 1998; Cory and Adams, 2002). The family comprises over 20 members that share one or more of the Bcl-2 Homology functional domains 1-4 (FIG. 1). Bcl-2 family member proteins can be broadly divided into two categories depending on their ability to either protect or promote apoptosis. The anti-apoptotic Bcl-2 proteins include Bcl-2 itself, Bcl-XL, Bcl-w, MCl-1, A1 and Diva; the pro-apoptotic Bcl-2 protein category is much larger and includes members such as Bax, Bak, Bik, Bid and Bim. The pro-apoptotic Bcl-2 proteins can be further divided into two classes based on of the number of BH domains each member contains. The multi-domain, pro-apoptotic Bcl-2 proteins can directly promote the initiation of apoptosis and include Bax, Mtd (Bok), Bak and Bcl-2-rambo; on the other hand, the BH3-only pro-apoptotic Bcl-2 proteins (e.g. Bik, Bad, Bim, Bik, Puma and Bcl-G) cannot directly promote apoptosis but rather act by associating with, and negating the action of, anti-apoptotic Bcl-2 proteins (Huang D C and Strasser A (2000); Bouillet P and Strasser A (2002a); Thomenius M J, et al., (2003)).

Bcl-2 proteins have been classically described as acting at the mitochondrion where they modulate the formation of the mitochondrial transition pore (MTP) and the subsequent release of apoptogenic factors such as cytochrome C, Smac/Diablo, AIF, HSP60, HtrA2/Omi and endonuclease G from the mitochondrial intermembrane space into the cytoplasm (Yang J, et al., (1997); Kroemer G (1998); Antonsson B (2001)). In particular, the release of cytochrome C activates Apaf-1, which converts procaspase-9 into active caspase-9, which in turn cleave-activates caspase-3, thus initiating the proteolytic apoptotic cascade (Allen et al., 1998; Mancini M, et al., (1998); Gross A, et al., (1999); Susin S A, et al., (1999)).

While some Bcl-2 family members are permanently inserted into the mitochondrial outer membrane, others have been found in the cytosol or associated with other subcellular compartments (Germain M and Shore G C (2003)). For example, the pro-apoptotic Bad, Bax and Bim are found in the cytosol or associated with element of the cytoskeleton (Bim), but relocate to the mitochondrion in response to apoptotic stimuli (Wolter K G, et al., (1997); Goping I S, et al., (1998); O'Connor L, et al., (1998); De Giorgi F, et al., (2002); Marani M, et al., (2002); Yamaguchi T, et al., (2003); Yuen A R and Sikic B I (2000)). On the other hand, Bax and Bak can also be targeted to the ER where they can initiate apoptosis through modulation of Ca++ release (Chandra J, et al., (2002); Zong W X, et al., (2003)). In addition to the mitochondrion, Bcl-2 also localizes to the cytosolic membranes of the ER and the nuclear envelope (Akao Y, et al. (1994); Wang Z H, et al. (1999); Germain and Shore, 2003). While Bcl-2 at the ER may modulate Ca++ storage (Distelhorst C W and Shore G C (2004)), the function of Bcl-2 at the nuclear envelope has been poorly investigated and is unclear.

It is estimated that in 2004, 33,440 Americans will develop leukemia and 23,300 will succumb to the disease (SEER Cancer Statistics Review, 1975-2001, National Cancer Institute. Bethesda, Md., http://seer.cancer.gov/csr/1975_{—}2001/2004). Most forms of leukemia, particularly relapsing acute myeloid leukemia (AML), are known to develop resistance to chemotherapeutic drugs, a result often associated with high levels of Bcl-2 expression (Campos L, et al., (1993); Bradbury D A and Russell N H (1995); Porwit-MacDonald A, et al. (1995); Reed, et al., (1995); Konopleva et al., (1999); Konopleva M, et al. (2002b)).

Development of drug resistance is a significant problem because it negates the benefit of the only effective therapy available and inevitably underscores the fatal outcome of leukemia. The urgent need for an effective solution to this therapeutic problem is illustrated by the extensive amount of research devoted to understanding drug resistance in leukemias (Campos L, et al (1994); Maung Z T, et al., (1994); Ruvolo P P, et al., (1998); Andreeff M, et al., (1999); Pepper C, et al., (1999); Konopleva M, et al. (2000); Carter B Z, et al., (2003a); Cohen-Saidon C, et al., (2003); Jiffar T, et al., (2004)). New experimental therapeutic approaches for sensitive cancer types include the suppression of Bcl-2 expression (Campos et al., 1994; Kitada S, et al., (1994); Cotter F E, et al., (1999); Gleave M E, et al., (1999); Waters J S, et al., (2000); Ziegler A, et al., (2000); Klasa R J, et al., (2002); Frankel S R (2003); Nahta R and Esteva F J (2003); Noguchi S, et al., (2003)), or antagonizing its protective action (Wang J L, et al., (2000a); Wang J L, et al., (2000b); Feng W Y, et al., (2003)). Both strategies, however, require concomitant chemotherapy and/or functional pro-apoptotic Bcl-2 family members (e.g., Bax) to be successful (Wei M C, et al., (2001); Bouillet P and Strasser A (2002b); Klasa et al., 2002; Marani et al., 2002; Panaretakis T, et al., (2002); Tanabe K, et al., (2003)). Therefore, the efficacy of such approaches may be limited (Reed J C (1997); Zong W X, et al., (2001); Juin et al., 2004; Pommier et al., 2004).

Acquired resistance to chemotherapeutic drugs is the most important cause of treatment failure and fatal outcome of aggressive human cancers such as relapsing acute myeloid leukemia (AML). While active efflux of drugs is among the best characterized mechanisms of multi-drug resistance in cancer cells, it is now apparent that independent downstream cellular responses to chemotherapeutic agents determine the outcome of therapy. An overwhelming body of evidence points to the fundamental role played by the Bcl-2 family of protein modulators of cell apoptosis in mediating resistance to chemotherapy-induced apoptosis of relapsing leukemias. Indeed, overexpression of Bcl-2 prevents apoptosis induced by the most common chemotherapeutic agents. In particular, alterations of Bcl-2 expression have been described in AML, where high levels of Bcl-2 are associated with poor response to chemotherapy and shortened survival. Consequently, the latest experimental therapies are aimed at overcoming drug resistance by either suppressing Bcl-2 expression or antagonizing its protective function. Both approaches, however, require concomitant chemotherapy and/or functional pro-apoptotic Bcl-2 family members to be successful. Therefore, the effectiveness of these strategies may be limited.

Recent evidence that Bcl-2 itself can function as a pro-apoptotic protein has suggested a powerful alternative approach to eliminate drug-resistant Bcl-2-overexpressing cancer cells. Specifically, utilizing Bcl-2's pro-apoptotic ability would overcome the problems mentioned above and kill Bcl-2-expressing cancer cells regardless of whether drug resistance is due to Bcl-2 or whether pro-apoptotic Bcl-2 family members are functional. Therefore, this novel approach would represent a significant therapeutic advantage.

Better pharmacological strategies are required to trigger Bcl-2-promoted apoptosis in drug resistant cancer cells.

Bcl-2 is targeted to the mitochondrion by the chaperoning action of the inherent calcineurin inhibitor FKBP38 (FIG. 2a) (Shirane M and Nakayama K I (2003)). Targeting of recombinant FKBP38 away from the mitochondrion alters Bcl-2 sub-cellular distribution and negates Bcl-2 protection.

There is still a significant gap in the current knowledge base on how Bcl-2 can be pharmacologically manipulated to promote apoptosis. This knowledge gap is significant because, until this information becomes available, it will not be possible to develop new effective drugs to selectively target high Bcl-2-expressing cancer cells.

### SUMMARY OF THE INVENTION

The present invention provides methods of screening a collection of compounds or libraries thereof to identify compounds that disrupt Bcl-2/FKBP38 binding and thereby induce apoptosis in a cell.

This screening method is based on the observation that if the binding of Bcl-2 with the carrier protein FKBP38 is disrupted, Bcl-2 is misplaced and associates with the nuclear envelope, where Bcl-2 actively promotes apoptosis by decreasing transcription factor entrance into the nuclear compartment. The present invention is based on a novel pro-apoptotic function of nuclear localized Bcl-2. Specifically, using compounds that disrupt BH4 (Bcl-2) domain mediated binding between Bcl-2 and FKBP38 will allow Bcl-2 to travel to the nucleus and kill the cell. The invention involves a method to manipulate Bcl-2 so as to kill malignant cells, while leaving normal cells unaffected.

Compounds selected in the screening method are those which trigger apoptosis in cancer cells that abundantly produce or overproduce Bcl-2. Compounds discovered by the claimed screening method find use as anticancer therapy in treating the development of a variety of malignancies where the cells either express or over-express Bcl-2 in a variety of cancer cells types which are well known in the art (Kirkin et al., (2004)).

This method involves the use of two lines of PC12 cells that have been stably transfected with Bcl-2 conjugated with an indicator or marker such as the Green Fluorescent Protein (PC12-Bcl-2-GFP) or GFP alone (PC12-GFP). Such transfected cells will be further stably transfected with FKBP38 conjugated with an indicator or marker such as Cyan Fluorescent Protein (FKBP38-CFP).

Both cell lines are exposed to appropriate concentrations of the test compounds. Twenty-four to 48 hr later the extent of cell death is measured. Test compounds that induce cell death in PC12-Bcl-2 but not in PC12-Vector are will be selected as compounds able to induce Bcl-2-promoted apoptosis.

Therefore, this simple method will allow rapid and high throughput screening of test compounds that result in Bcl-2-promoted cell death.

Further testing of active compounds can be done by treating larger cultures (T-25 flasks) of stably transfected PC12 cells with such compounds and then measuring the appearance of nuclear Bcl-2 by western blot applied to cytosolic and nuclear protein extracts from treated cells. This method allows the rapid identification of lead compound for targeting and killing cells expressing Bcl-2 while leaving cells that lack Bcl-2 expression unaffected.

The invention is directed towards a method of screening compounds that disrupt Bcl-2/FKBP38 binding and thereby induce apoptosis. The invention is also directed towards a method of promoting apoptotic cell death in Bcl-2 producing cells or tissues by contacting said cells or tissues with a sufficient amount of BH4 peptide or mimetic thereof to inhibit of Bcl-2/FKBP38 binding. Additionally, the invention is directed towards a method of purging malignant, Bcl-2 producing cells from a mixed population of cells, by contacting the mixed population with a sufficient amount of BH4 peptide or a mimetic thereof to disrupt Bcl-2/FKBP38 binding and trigger apoptosis in Bcl-2 producing cells. The mixed population of cells can be in or from an individual.

### BRIEF DESCRIPTION OF THE FIGURES

FIG. 1. A schematic representation of the domain structure of Bcl-2.

FIGS. 2A and B. A: Functional domains of the FKBP38 protein (top); a proposed model for FKBP38 mediated localization of Bcl-2 to the mitochondria (bottom). B: The expected structure of the Bcl-21-30 peptide, which includes the Bcl-2's BH4 domain (AA 11-25) and is henceforth referred to as BH4 peptide.

FIG. 3: Multi-parameter flow cytometry of cells from 3 AML patients that were induced to undergo apoptosis.

FIG. 4: Flow cytometry measuring apoptotic cells from the nuclei of PC12 cells transiently transfected with Bcl-2-GFP.

FIG. 5: Flow cytometry measuring apoptotic cells from PC12 cells transiently transfected with Bcl-2-GFP with and without H₂O₂ treatment.

FIG. 6: PC12 cells transiently transfected with Bcl-2/YFP (or YFP as control) and 72 hr later cells undergoing apoptosis assayed by determining Annexin-V and propidium iodide (PI) reactivity using flow-cytometry.

FIG. 7: Western blot assay of protein fractions from PC12 cells transiently transfected with vector, Bcl-2, or Bcl-2ΔBH4.

FIG. 8: Bcl-2-transfected PC12 cells were stained for mitochondria (using MitoTracker) and nuclei (using DAPI) and then observed with a confocal microscope.

FIG. 9: Western blot of protein fractions from PC12 cells transiently transfected with vector, Bcl-2, or Bcl-2ΔBH4 in the presence or absence of co-transfected FKBP38.

FIG. 10: PC12 cells were transiently transfected with Bcl-2 or Bcl-2ΔBH4 in the presence or absence of co-transfected FKBP38. 48 hr later, nuclear fractions were prepared and analyzed by Western blot for the presence of Bcl-2, Bcl-2ΔBH4 and FKBP38.

FIG. 11: Western blot of protein fractions from either transiently or stably-transfected PC12 cells.

FIG. 12: Western blot of protein fractions from PC12 cells stably-transfected with an inducible Bcl-2 expression system.

FIG. 13: Luciferase activity of PC12 cells transiently transfected with either vector alone or Bcl-2 DBH4.

FIG. 14: Flow cytometry measuring apoptotic cells from either transiently or stably-transfected PC12 cells.

FIG. 15: PC12 cells were transiently transfected with Bcl-2 or Bcl-2ΔBH4 in the presence or absence of co-transfected FKBP38. All cells were further transfected with renilla luciferase. 48 hr later, the amount of renilla luciferase (indicative of the number of surviving transfected cells) was measured using a luminescence activity assay.

FIG. 16: Flow cytometry measuring apoptotic cells from PC12 cells transiently transfected with Bcl-2 or vector alone.

FIG. 17: PC12 cells were transiently transfected with Bcl-2/YFP. After 48 hr, the nuclei were isolated and analyzed by flow cytometry.

FIG. 18: Flow cytometry measuring apoptotic cells from PC12 cells transiently transfected with Bcl-2-GFP with and without Ca²⁺ treatment.

FIG. 19: Cell survival was measured in PC12 cells stably expressing vector or Bcl-2. These two cell lines were transiently co-transfected with luciferase and either vector, BH4 domain, or BH4Gly.

FIG. 20: Cell survival was measured in PC12 cells transiently co-transfected with Bcl-2 or vector, and either vector, BH4 or BH4Gly.

FIG. 21A: Proposed mechanism of initiation of apoptosis induced by the association of Bcl-2 with the nuclear envelope as a consequence of transient high expression of Bcl-2 and overwhelming of FKBP38 capacity.

FIG. 21B: Association of Bcl-2 with the nuclear envelope and subsequent apoptosis promoted by a BH4 peptide via competition of Bcl-2/FKBP38 complex formation.

FIG. 22: Bcl-2/FKBP38 cotransfected PC12 cells were stained with antibodies for Bcl-2 and FKBP38 and for nuclei (using DAPI) and then observed with a confocal microscope.

FIG. 23: Bcl-2 or Bcl-2ΔBH4 transfected PC12 cells were stained for Bcl-2 and for nuclei (using DAPI) and then observed with a confocal microscope.

FIG. 24: Human leukemia cells (HL-60) were transiently transfected with a BH4 peptide-expressing vector, with the inactive control (BH4Gly) or with empty vector (Flag). The extent of cell death was assessed 48 after transfection as release of LDH in the cell culture medium.

FIG. 25: Western blot of PC12 cells transfected with an inducible Bcl-2 expression system.

FIG. 26: PC12 cells were transiently transfected with Bcl-2 in the presence of co-transfected FKBP38. Total cell homogenates were immunoprecipitated with an anti-Bcl-2 antibody to pull down Bcl-2 and analyzed by Western blot probing for FKBP38, Bcl-2 and β-actin.

FIG. 27: PC12 cells were transiently transfected with Bcl-2 or Bcl-2ΔBH4 in the presence of co-transfected FKBP38. Total cell homogenates were immunoprecipitated with an anti-HA antibody to pull down FKBP38 (FKBP38 is tagged with HA) and analyzed by Western blot probing for FKBP38 or Bcl-2.

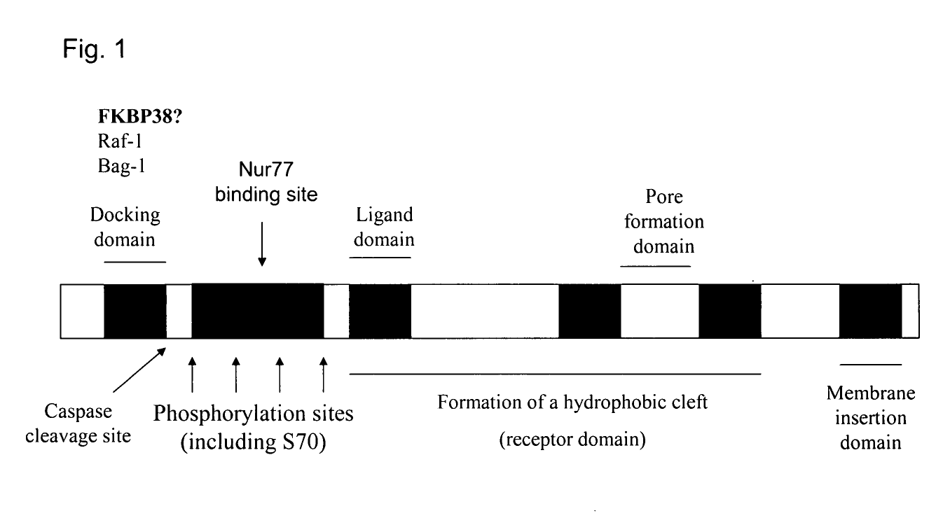

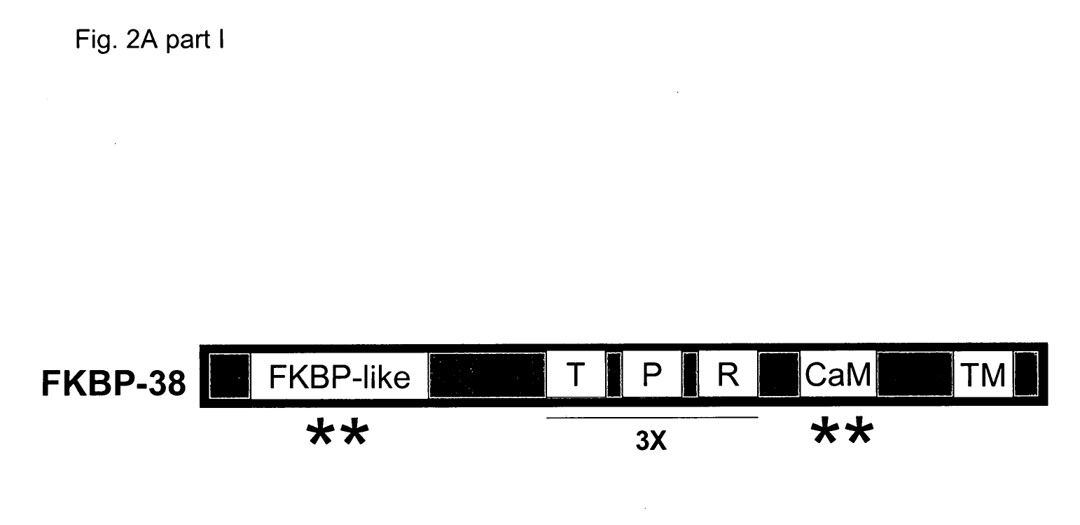

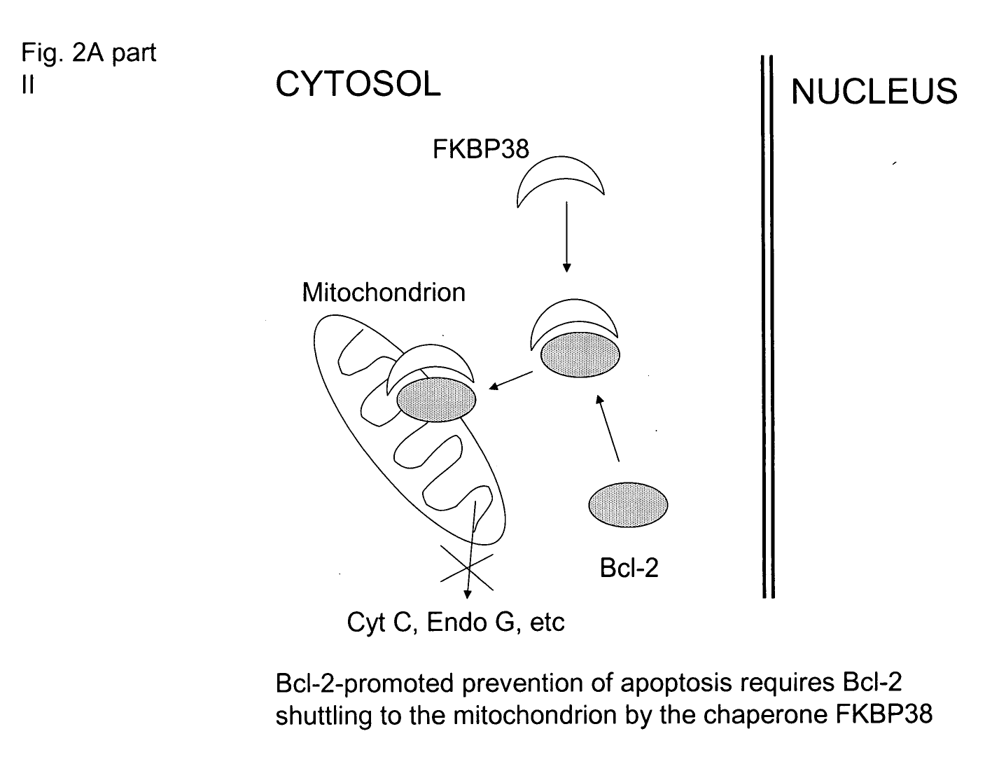

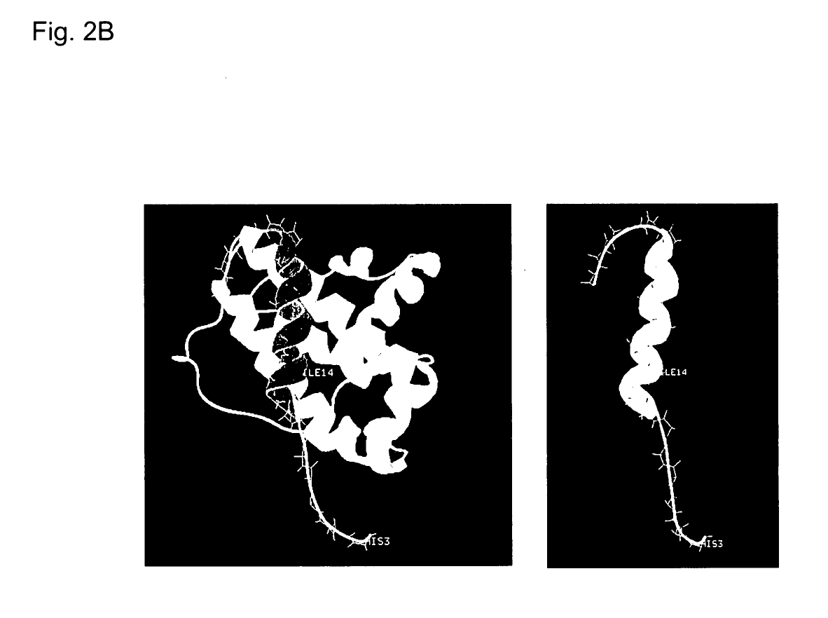

### DETAILED DESCRIPTION OF THE INVENTION

The present invention provides a method of screening for compounds that interrupt Bcl-2/FKBP38 binding via the BH4 domain, and which trigger Bcl-2 promoted cell death. A cell model system has been developed which contains peptides of the Bcl-2 BH4 domain to assay for the ability of tested compounds to interfere with Bcl-2/FKBP38 binding, causing Bcl-2 to accumulate at the nucleus and subsequently induce apoptosis.

As shown herein, interfering with Bcl-2/FKBP38 binding promoted apoptosis. Thus, targeting the interaction between Bcl-2 and FKBP38 is an effective way to modulate the anti-apoptotic function of Bcl-2.

Compounds selected in the screening method are those which trigger apoptosis in cells that abundantly produce or overproduce Bcl-2. The compounds will be screened for their ability to effectively mimic the Bcl-2/FKBP38 binding-antagonizing effect of the Bcl-2 BH4 domain peptide while being therapeutically manageable. By way of example but not limitation, these cells include leukemia, malignant melanoma, colon cancer, hormone-refractory breast cancer. Compounds discovered by the claimed screening method find use as anticancer therapy in treating the development of a variety of malignancies dependent on over-expressed Bcl-2 from a variety of cancer cells types which are well known in the art (Kirkin et al. (2004)). Tumor types which are targeted by the compounds discovered by the claimed screening method include, but are not restricted to: acute lymphoblastic leukemia, precursor B-lymphoblastic leukemia/lymphoma, diffuse large B-cell lymphoma, liposarcoma, squamous cell carcinoma, meningioma, breast carcinoma, gliobastoma, Hodgkin lymphoma, ependymoma, gastrointestinal stromal tumors, leukemia, melanoma, colon cancer, and hormone-refractory breast cancer, including without limitation malignancies such as prostate, colorectal, lung, gastric, renal neuroblastoma, non-Hodgkin's lymphoma, acute leukemia, and chronic leukemia (Kirkin et al. (2004)).

Several types of metastatic cancer cells become resistant to chemotherapy with fatal outcomes, partially because they express high levels of Bcl-2 and therefore they are protected from the cytotoxic effects of chemotherapeutic drugs. The present invention involves a method of promoting apoptotic cell death in Bcl-2 producing cells or tissues by contacting the cells or tissues with a compound to inhibit the binding of Bcl-2 and FKBP38. This inhibition or disruption of Bcl-2/FKBP38 binding triggers apoptosis in resistant cells, thus eliminating such cancer cells regardless of whether Bcl-2 is the main factor providing resistance to chemotherapy. The invention is effective in the absence of chemotherapy, and can also be used in non-operable cancers.

This invention uses Bcl-2 as a pro-apoptotic agent that can be used to kill cancer cells regardless of whether Bcl-2 played a role in their malignancy and in cooperation with or independently of chemotherapy.

### Method for Screening Apoptotic Compounds: Cell-Based Screening Assays

In one embodiment of the invention, Pheochromocytoma (PC12) cells can be used as a cell model for screening compounds that interrupt the interaction between Bcl-2 and FKBP38. PC12 cells do not express endogenous Bcl-2 (Massaad C A and Taglialatela G (2003)) and yet Bcl-2 is functional once expressed in PC12 cells, as shown by several studies in the past which employed PC12 cells stably transfected with Bcl-2 (Sato N, et al., (1994); Tyurina Y Y, et al., (1997); Okuno S, et al., (1998); Deng G M, et al., (1999); Kaufinann J A, et al., (2003); Song Y S, et al., (2004)).

In one embodiment of the invention, two lines of PC12 cells were used that had been stably transfected with Bcl-2 conjugated with Green Fluorescent Protein (PC12-Bcl-2-GFP) or GFP alone (PC12-GFP). Therefore, these stably transfected cell lines are identical except that one expresses Bcl-2 while the other does not. Such transfected cells were further stably transfected with FKBP38 conjugated with Cyan Fluorescent Protein (FKBP38-CFP). Stably transfected PC12 cell stock lines can be stored frozen in liquid nitrogen and maintained indefinitely.

Both cell lines were thawed as needed and plated into 96-well plates and exposed to appropriate concentrations of the test compounds (nM to mM, depending on test compounds). Twenty-four to 48 hr later the extent of cell death was measured by assaying lactate dehydrogenase (LDH) release in sampled aliquots of the culture medium. Compounds that induce cell death (significant increase in LDH release) in PC12-Bcl-2 but not in PC12-Vector are selected as being able to induce Bcl-2-promoted apoptosis.

At the same time (and in the same 96 well plate used for LDH determinations), fluorescence resonance energy transfer (FRET) due to the binding of the transfected Bcl-2-GFP to the transfected FKBP38-CFP (and thus to energy transfer between adjacent GFP and CFP brought about by the formation of the Bcl-2/FKBP38 complex) was measured by a microplate fluorimeter. Disappearance of FRET energy in response to test compounds treatment indicates that the test compound had disrupted Bcl-2/FKBP38 binding.

Therefore, this method allows rapid and high throughput screening of test compounds that disrupt Bcl-2/FKBP38 binding (FRET measurements) and result in Bcl-2-promoted cell death (LDH measurements). In one aspect the method of the invention treats larger cultures (T-25 flasks) of stably transfected PC12 cells with such compounds and then measures the appearance of nuclear Bcl-2 by western blot applied to cytsolic and nuclear protein extracts from treated cells.

This method allows the rapid identification of lead compounds to be developed for targeting cells expressing Bcl-2 while leaving cells not expressing Bcl-2 unaffected.

Non-Cell Based Screening Assays

Non-limiting examples of non-cell based methods for screening compounds that interfere with Bcl-2/FKBP38 binding and trigger nuclear Bcl-2-promoted cell death is, for example, by the use of screening phage-display peptide libraries or libraries of synthetic compounds. (Kay et al., (2001); Swevers et al., (2004). Methods in assay development and high throughput screening and combinatorial chemistry are available to those skilled in the art for cell-based as well as non-cell based screening of interactions between proteins, ligands, and nucleic acids. Also see resources available at commercial suppliers of high-throughput screening materials and equipment (e.g. Sigma-Aldrich Company).

Detection of Apoptosis by Flow Cytometry

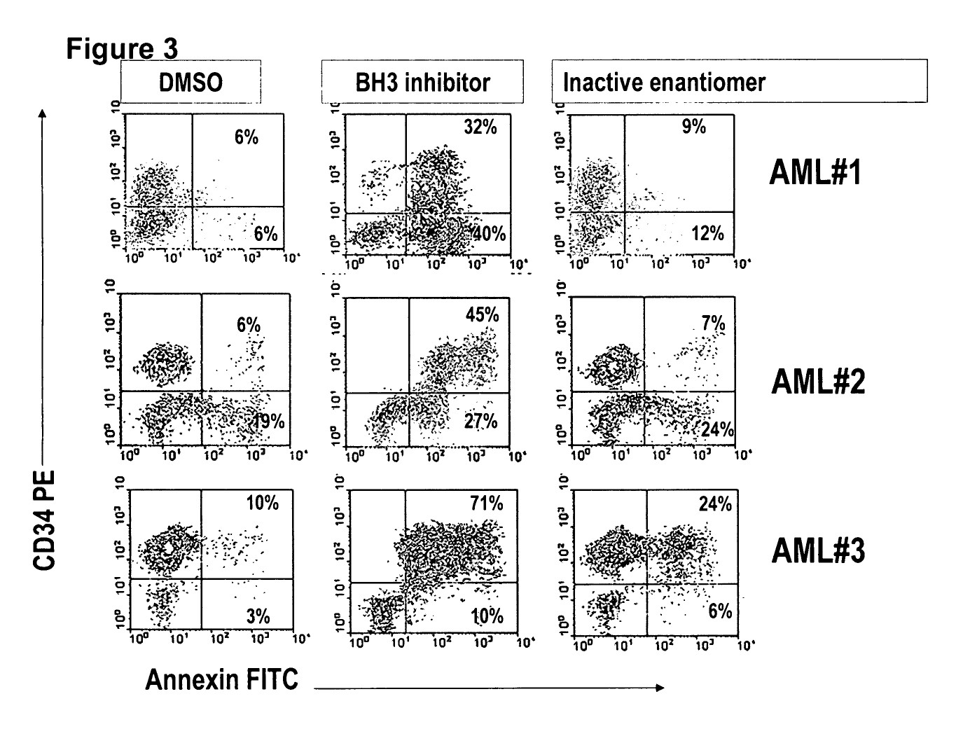

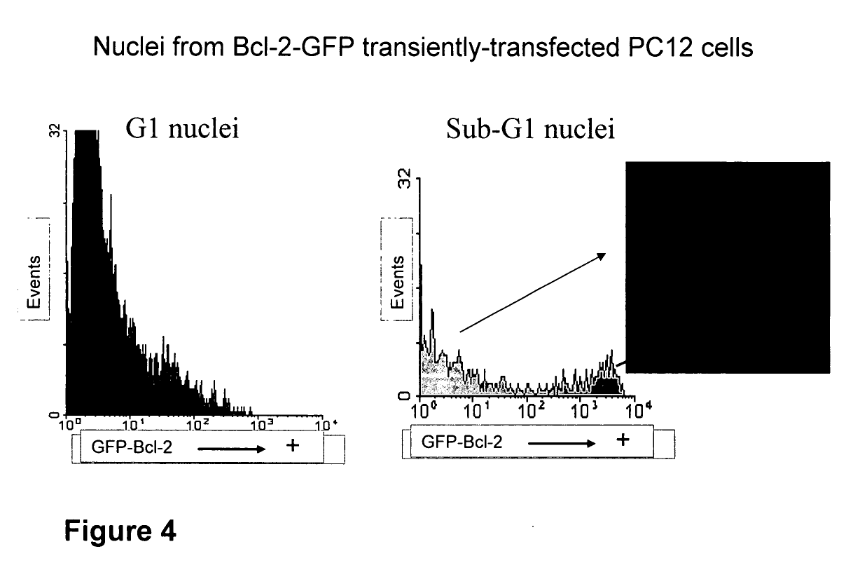

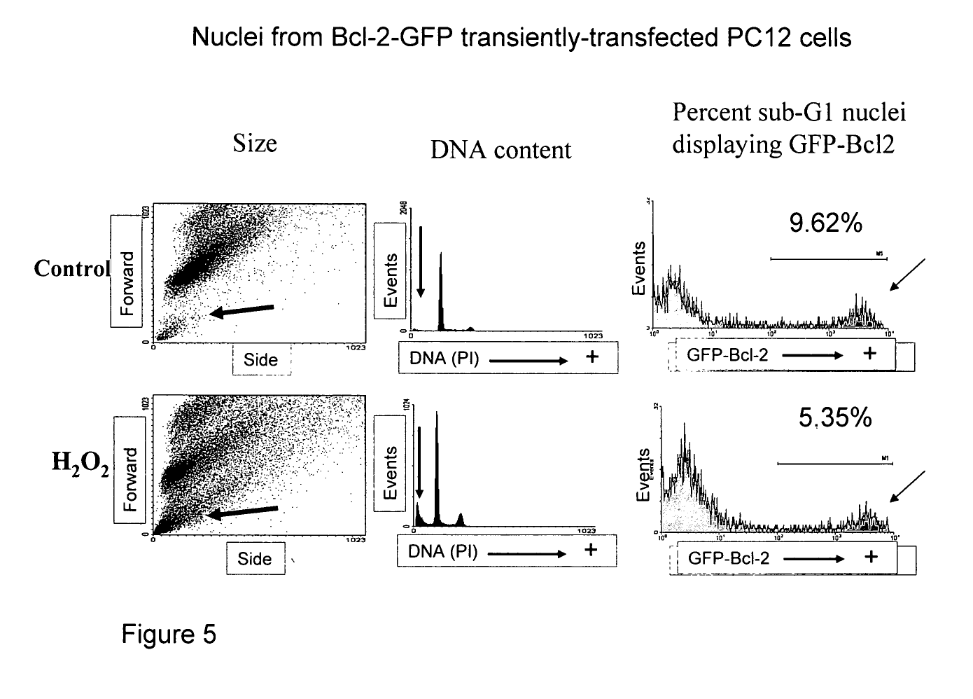

FIG. 3 shows the results of flow cytometry, which allows simultaneous detection of DNA and multiple antigens in sorted cells. This technique allows for detection of Bcl-2 associated with apoptotic nuclei (as e.g. illustrated in FIGS. 4 and 5), but also to detect apoptosis (by FITC Annexin-V/PI staining) in selected primary AML cells and CD34±38-normal stem cells. FIG. 3 shows multi-parameter flow cytometry applied to samples from experiments detecting apoptosis in primary cells from 3 separate AML patients. Multi-parameter flow cytometry allows simultaneous assessment of the presence of Annexin-V staining (apoptotic cells right quadrants) and CD34 antigen (progenitor cells, upper quadrants).

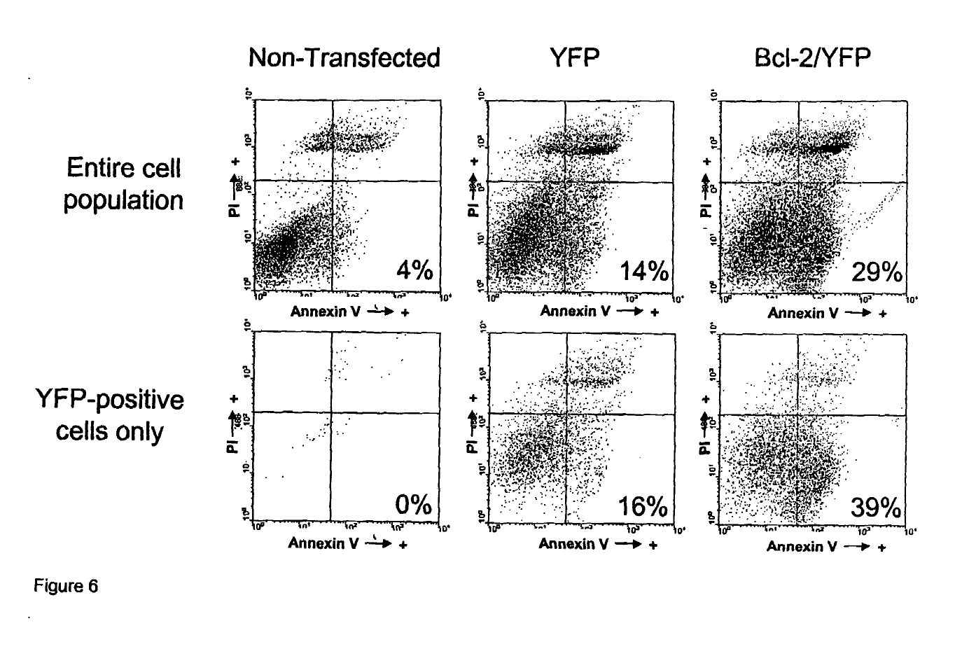

FIG. 6 shows PC12 cells that were transiently transfected with Bcl-2/YFP (or YFP as control) and 72 hr later cells undergoing apoptosis were assayed by determining Annexin-V and propidium iodide (PI) reactivity using flow-cytometry. In this standard assay, apoptotic cells are positive for Annexin-V and negative for PI staining and will therefore be located in the lower-right quadrant. Note the higher percent of apoptotic cells in Bcl-2 transfected cells as compared to YFP transfected cells. The percent of apoptotic cells in the Bcl-2 transfected group is even higher when only transfected cells (YFP-positive) are analyzed.

### Selective Purging of Malignant, Bcl-2 Producing Cells

A method of the invention is the use of domain interference to effectively induce apoptosis in a Bcl-2 dose-dependent fashion and not affect cells that are devoid of Bcl-2 expression. This feature allows for the therapeutic development of the BH4 domain interference approach, which targets high Bcl-2-expressing AML cells and leaves Bcl-2-negative hematopoietic progenitor stem cells unaffected.

### EXAMPLE

### FKBP38 Removes Bcl-2 from the Nucleus

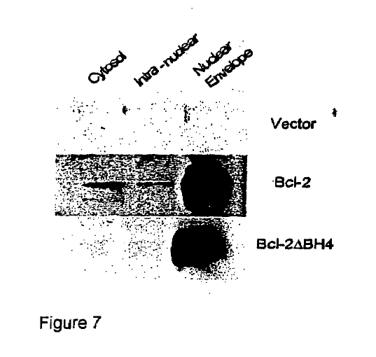

In the experiment shown in FIG. 7, PC12 cells were transiently transfected with Bcl-2 or Bcl-2ΔBH4 (a Bcl-2 deletion mutant lacking the N-terminal 30 amino acids, including the 11-25 BH4 domain) and the presence of Bcl-2 in the cysosolic, intranuclear and nuclear envelope protein faction assayed by western blot. Bcl-2 was localized in the cytosolic fraction (containing mitochondria) but not in the intra-nuclear fraction. There was a predominant presence of Bcl-2 at the nuclear envelope. Similarly, Bcl-2ΔBH4 was localized at the nuclear envelope and, at variance with Bcl-2, it was absent from the intra-nuclear or cytosolic fractions. The complete absence of cytosolic (mitochondrial) Bcl-2ΔBH4 suggests that Bcl-2ΔBH4 failed to bind FKBP38 altogether.

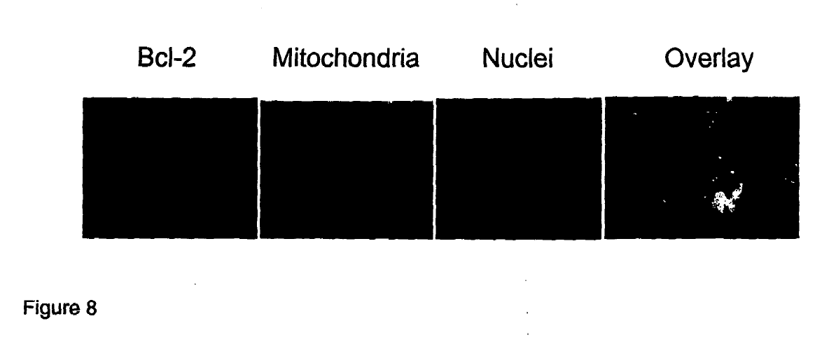

In FIG. 8, Bcl-2 transfected PC12 cells were stained for mitochondria (using MitoTracker) and nuclei (using DAPI) and then observed with a confocal microscope. Note that while Bcl-2 ringed the nucleus, mitochondria did not. Indeed, Bcl-2 co-localized with mitochondria in the cytosolic area, as expected. Thus, the perinuclear presence of Bcl-2 is not due to mitochondrial clustering around the nucleus. Rather, it is a phenomenon independent of Bcl-2 association with the mitochondria.

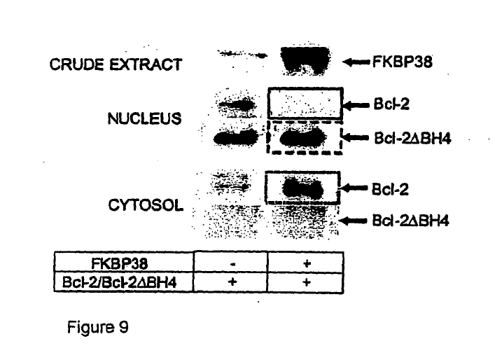

The ability of FKBP38 to remove (or prevent) nuclear association of Bcl-2 or Bcl-2ΔBH4 was measured (FIG. 9). The results show that transient over-expression of FKBP38 removes (or prevents) nuclear presence of co-expressed Bcl-2, but not Bcl-2ΔBH4. In this experiment, PC12 cells were co-transfected with Bcl-2 or Bcl-2ΔBH4 in the presence (right lanes) or absence (left lanes) of co-transfected FKBP38 (shown by the western blot on crude protein extracts on top). The western blot in the middle of FIG. 9 was performed on total nuclear protein extracts (intranuclear+envelope) from these cells and shows that overexpressed FKBP38 removes Bcl-2 (boxed in solid lines) but not Bcl-2ΔBH4 (boxed in dashed lines) from the nucleus and directs it to the cytosol (boxed in dotted lines), as shown in the western blot at the bottom of FIG. 9, which was performed on cytosolic protein extracts from these cells.

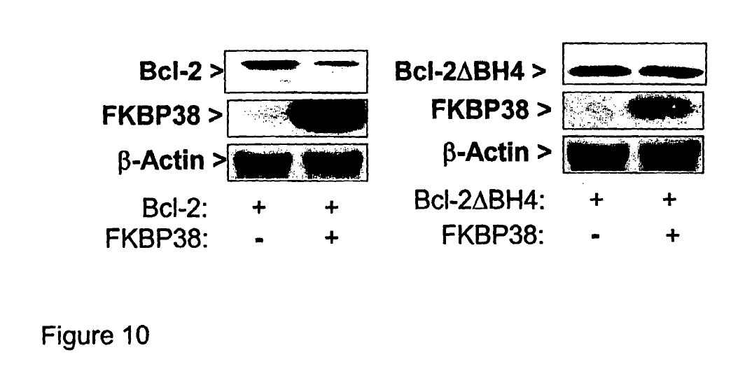

Similarly, PC12 cells were transiently transfected with Bcl-2 or Bcl-2ΔBH4 in the presence or absence of co-transfected FKBP38. 48 hr later, nuclear fractions were prepared and analyzed by Western blot for the presence of Bcl-2, Bcl-2ΔBH4 and FKBP38. (FIG. 10) Co-expression of FKBP38 reduced the nuclear presence of Bcl-2 but not of Bcl-2ΔBH4. Lack of Bcl-2ΔBH4 removal from the nucleus by FKBP38 suggests that the BH4 domain is necessary for Bcl-2/FKBP38 interaction. Thus, Bcl-2 binds to FKBP38 via Bcl-2's BH4 domain and lack of Bcl-2/FKBP38 binding resulted in Bcl-2 nuclear misplacement and apoptosis.

### Nuclear Bcl-2 Expression Induces Apoptosis

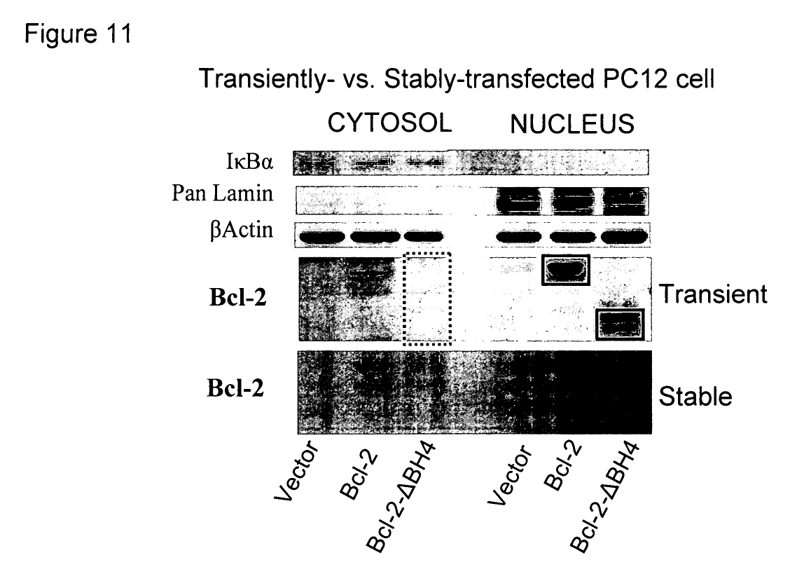

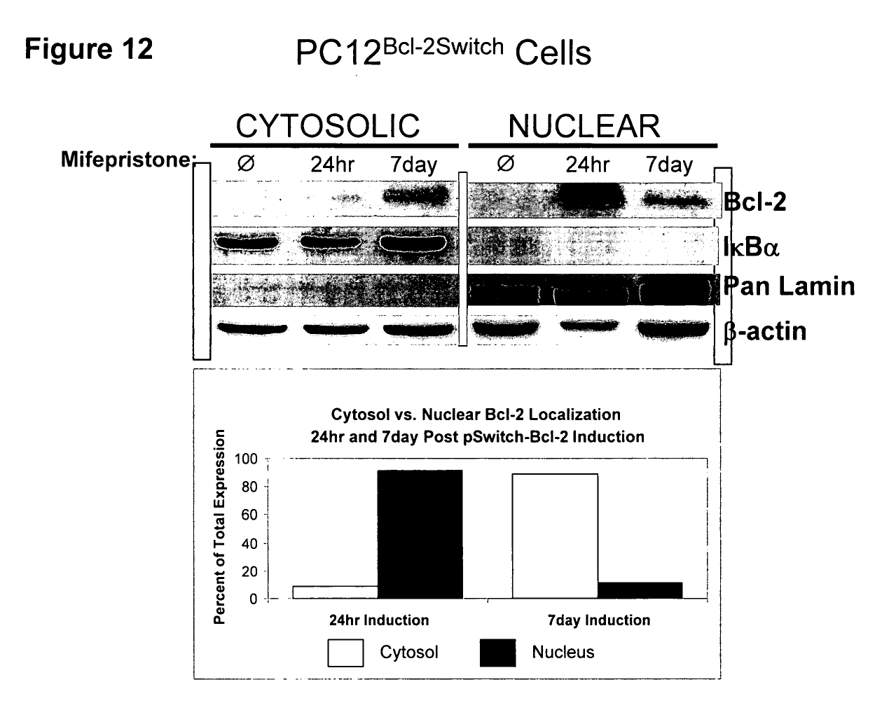

Association of Bcl-2 with the nucleus is sufficient to initiate apoptosis, independently of mitochondrial integrity and caspase-3 activation. Cells expressing nuclear Bcl-2 or Bcl-2ΔBH4 were selected against during establishment of stable transfection (FIG. 11). PC12 cells were transfected transiently (48 hr) or stably (antibiotic selection>1.5 months) with Bcl-2 or Bcl-2ΔBH4. Cytosolic and nuclear protein extracts were prepared and analyzed by western blot for expression and subcellular localization of Bcl-2 (and the smaller, truncated Bcl-2ΔBH4). Blots were re-probed to detect IkBa (a cytosolic marker), pan lamin (a nuclear marker) and β-actin to control for subcellular fraction purity and equal protein loading. Both Bcl-2 and Bcl-2ΔBH4 were detected in the nuclear fraction of cells transiently transfected (boxed in solid lines) but not in the nuclear fractions of stably transfected cells. It appeared that cells expressing Bcl-2 (or Bcl-2ΔBH4) in the nucleus died during establishment of long-term stable transfection. However, caution should be exercised when comparing stable vs. transient transfected cells since they are treated differently, stably-transfected cells have undergone long-term selection. To address this potential concern, we determined Bcl-2 subcellular distribution in PC12 Bcl-2-Switch cells, which are cells stably transfected with an inducible Bcl-2 expression system (GeneSwitch, Invitrogen) that can be turned on by treating cells with mifepristone. FIG. 12 shows that Bcl-2 expression was induced in the same batch of PC12 Bcl-2-Switch cells for either 24 hr or 7 days. At 24 hr, there was prevalent Bcl-2 expression in the nucleus as compared to the cytosol. However, at 7 days the converse was true. Also, overall Bcl-2 expression levels at 7 days were reduced as compared to 24 hr, suggesting perhaps that cells expressing high Bcl-2 levels had been eliminated. Collectively, the experiments shown in FIGS. 11 and 12 indicate that the nuclear presence of Bcl-2 was incompatible with long-term cell survival; hence, nuclear Bcl-2 killed these cells.

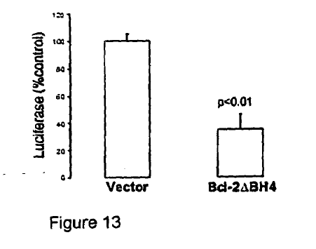

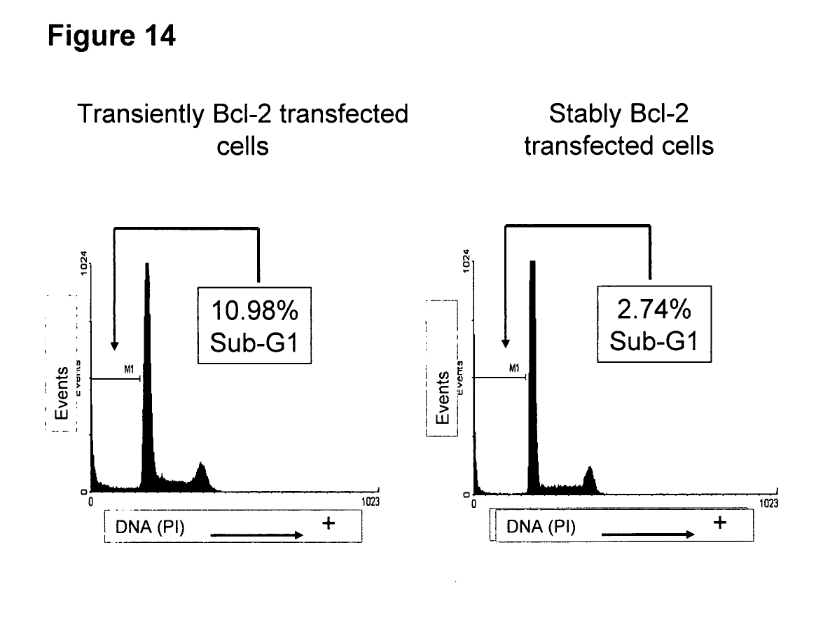

Cells stably expressing Bcl-2ΔBH4 were not obtained, consistent with the observation that Bcl-2ΔBH4 was almost entirely expressed in the nucleus upon transient transfection (note in FIG. 6 the absence of cytosolic Bcl-2ΔBH4 in transiently transfected cells, boxed in dotted line, and similar results in FIG. 7). Also consistent with this observation, transient transfection of Bcl-2ΔBH4 induced cell death (FIG. 13). In this experiment, Bcl-2ΔBH4 or the empty vector were co-transfected with renilla luciferase. Thus, luciferase levels were directly proportional to the number of live cells that received the transfected vectors. As shown in FIG. 8, there was a significantly lower luciferase level in the cells transfected with Bcl-2ΔBH4 as compared to cells transfected with the vector alone, indicating that Bcl-2ΔBH4 induced cell death. Furthermore, flow cytometry showed an increased presence of sub-G1 DNA (apoptotic DNA) in cells transiently transfected with Bcl-2 as compared to PC12 cells stably transfected (FIG. 14). In this experiment, the DNA content of cells transiently (left) and stably (right) transfected with Bcl-2 was analyzed by flow cytometry, revealing an increased amount of sub-G1 (apoptotic) DNA (indicated by the arrow) in cells transiently transfected (which carry nuclear Bcl-2) as compared to the cells stably transfected with Bcl-2 (which are devoid of detectable nuclear Bcl-2). While almost all of the stably transfected cells expressed the transgene (because of the experimental design), only 15-20% of the transiently transfected cells did and therefore the observed difference in subG1 (apoptotic) DNA was underestimated. Nonetheless, as stated above, caution should be exercised when comparing stable vs. transient transfected cells.

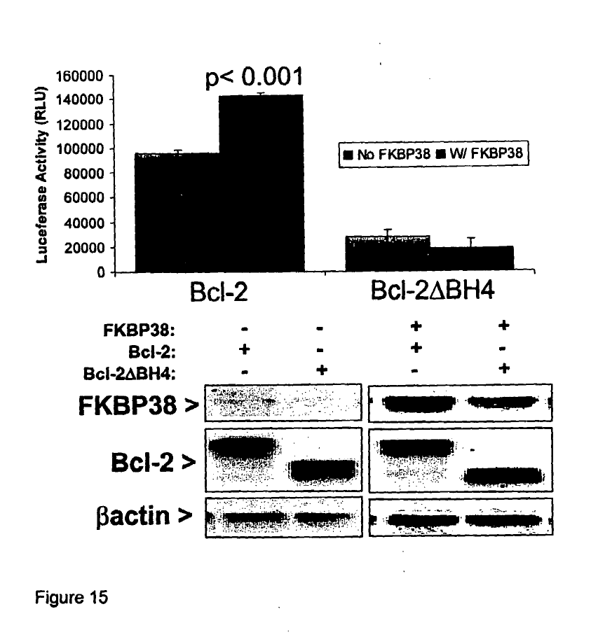

Co-expression of FKBP38 increased survival of Bcl-2-transfected PC12 cells but not in Bcl-2ΔBH4-transfected PC12 cells. FIG. 15 shows PC12 cells that were transiently transfected with Bcl-2 or Bcl-2ΔBH4 in the presence or absence of co-transfected FKBP38. All cells were further transfected with renilla luciferase. 48 hr later, the amount of renilla luciferase (indicative of the number of surviving transfected cells) was measured using a luminescence activity assay. Bcl-2ΔBH4-transfected cells survived less than Bcl-2-transfected cells (consistent with apoptosis induced by nuclear localization of Bcl-2, Bcl-2ΔBH4 is exclusively localized at the nucleus). Additionally, FKBP38 increased survival of Bcl-2-transfected cells (consistent with its ability to remove Bcl-2 from the nuclear compartment) but did not affect survival in Bcl-2ΔBH4-transfected cells (consistent with its failure to remove Bcl-2ΔBH4 from the nuclear compartment). Western blots at the bottom show expression of the transfected proteins in total protein extracts.

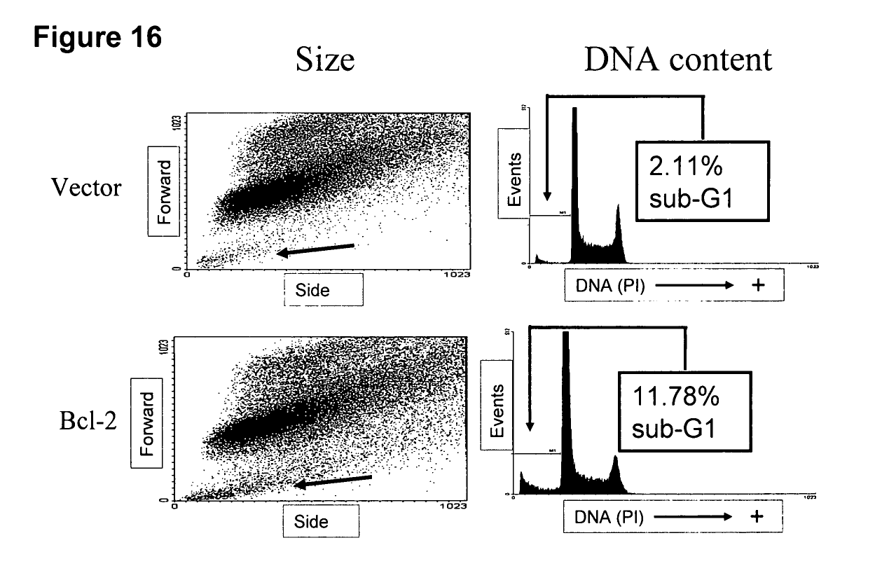

Selective occurrence of apoptosis in response to transient over-expression of Bcl-2 has been established (Uhlmann et al., 1998; Wang et al., 2001), and confirmed by the results reported in FIG. 16. In this experiment, cells were transiently transfected with either Bcl-2 or the empty vector control and DNA content determined by flow cytometry 48 hr later. There was a significant increase of sub-G1 DNA-containing (apoptotic, see arrows) nuclei in cells transfected with Bcl-2 as compared to vector. Therefore, the observed increased apoptosis was not a consequence of stress due to the transient transfection procedure; rather, apoptosis occurred specifically in response to transient transfection of Bcl-2. In addition, multiparameter flow cytometry applied to nuclei isolated from cells transiently transfected with Bcl-2 conjugated with a green fluorescent protein (GFP) tag, which allows detection of Bcl-2-GFP in sorted isolated nuclei, showed that Bcl-2-GFP was selectively associated with apoptotic nuclei (containing fragmented, sub-G1 DNA) but not with normal nuclei containing integer, G1 DNA (FIG. 4). This indicates that nuclear Bcl-2 was found selectively in cells undergoing apoptosis, which could happen only if Bcl-2 itself induced apoptosis once at the nucleus (otherwise significant levels of Bcl-2 would have also been observed in normal nuclei).

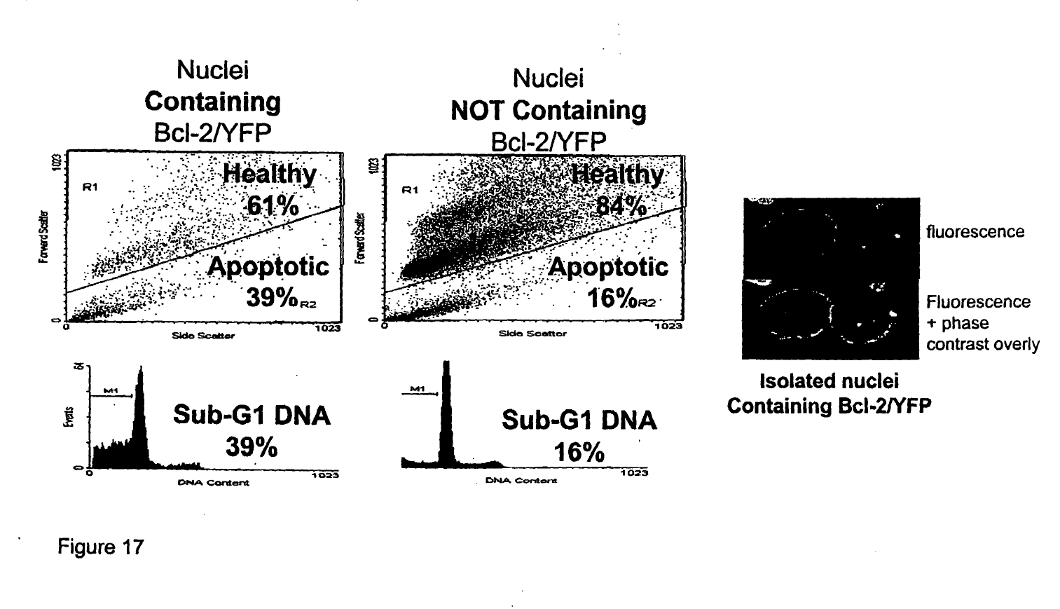

FIG. 17 shows PC12 cells that were transiently transfected with Bcl-2/YFP. After 72 hr, the nuclei were isolated and analyzed by flow cytometry. Analysis of nuclei containing Bcl-2/YFP (an example of such isolated nuclei is shown in the insert on the right) revealed 39% of apoptotic nuclei as compared to 16% observed in the nuclei that did not contain Bcl-2/YFP (upper panel). The same results were obtained when the amount of sub-G1 (apoptotic) DNA was determined in Bcl-2-containing vs. Bcl-2-non-containing nuclei (lower panel). Overall, these data indicate that induction of apoptosis after Bcl-2 transient transfection was associated with the presence of Bcl-2 in the nuclear compartment.

An alternative explanation would be that Bcl-2 relocates to the nucleus in response to apoptosis. To exclude this possibility, we conducted preliminary experiments where association of Bcl-2 with sub-G1 nuclei was determined by flow cytometry applied to nuclei isolated from cells transiently transfected with Bcl-2-GFP that were further exposed to H₂O₂ (an established pro apoptotic stimulus in PC12 cells (Kaufmann et al., 2003) (FIG. 5). Exposure of the cells to H₂O₂ induced apoptosis, as shown by the substantial increase in sub-G1 nuclei in treated cells as compared to untreated controls (arrows, left and center). If Bcl-2 associated with the nucleus in response to apoptosis, we would observe an increase in nuclear Bcl-2 in these H₂O₂-treated apoptotic cells. However, there was no increase in the percent of sub-G1 nuclei carrying Bcl-2-GFP in cells treated with H₂O₂ as compared to untreated cells. In fact, there was a paradoxical decrease, which is expected if one appreciates that the number of sub-G1 nuclei increased after H₂O₂ treatment without a concomitant increase of nuclear Bcl-2-GFP. Therefore, nuclear association of Bcl-2 was not a consequence of, but rather a cause of apoptosis.

### Method of Promoting Apoptotic Cell Death in BCL-2 Producing Cells

Association of Bcl-2 with the Nucleus Initiates DNA Fragmentation

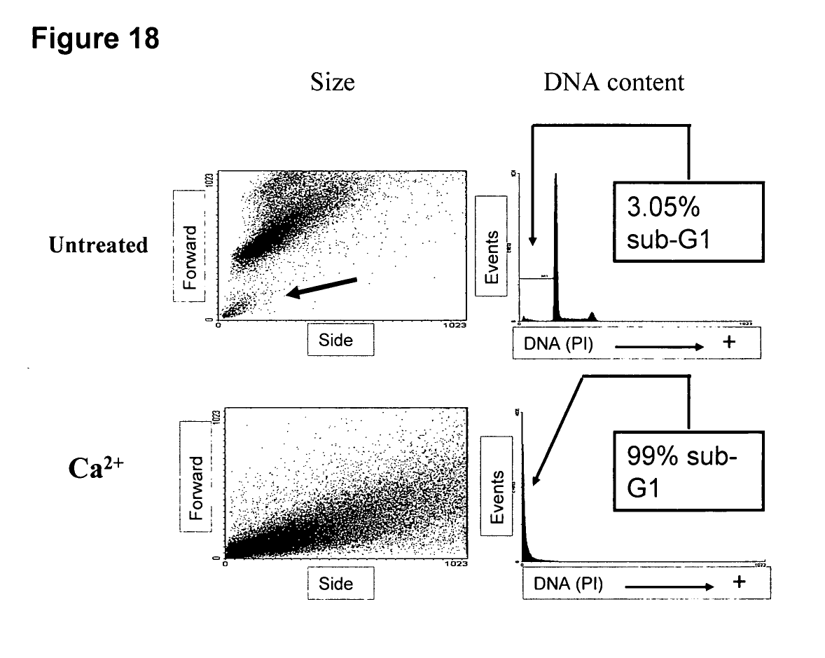

Our results show that nuclear Bcl-2 induced apoptosis by a novel mechanism, which was independent of caspase-3 and entails Ca²⁺-dependent initiation of DNA fragmentation. These experiments determined the extent to which exposure of isolated nuclei to recombinant Bcl-2 proteins was per se sufficient to initiate DNA fragmentation. Using a similar experimental design, excess Ca²⁺ in the incubation buffer triggered DNA fragmentation in isolated nuclei, which was detected by flow cytometry (FIG. 18).

### BH4 Domain Interference Induces Apoptosis in Cells Expressing Bcl-2

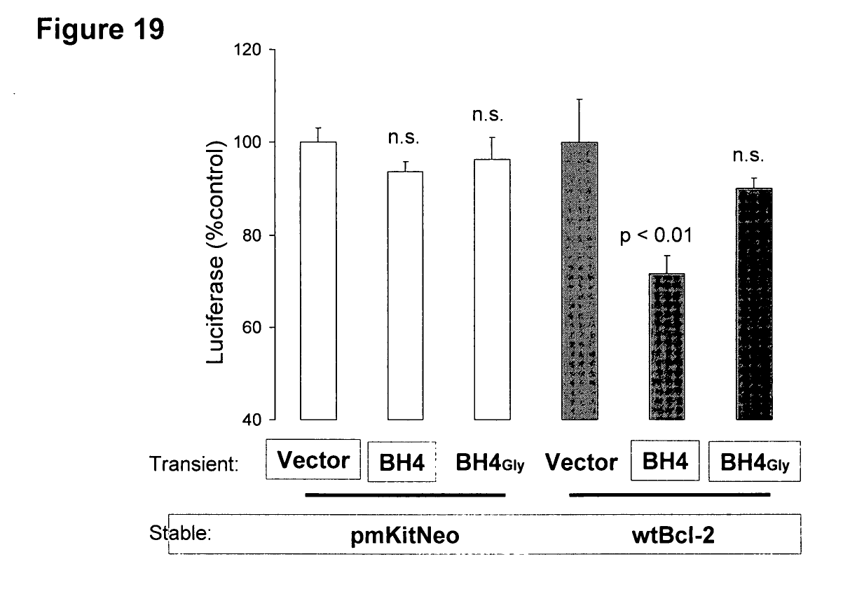

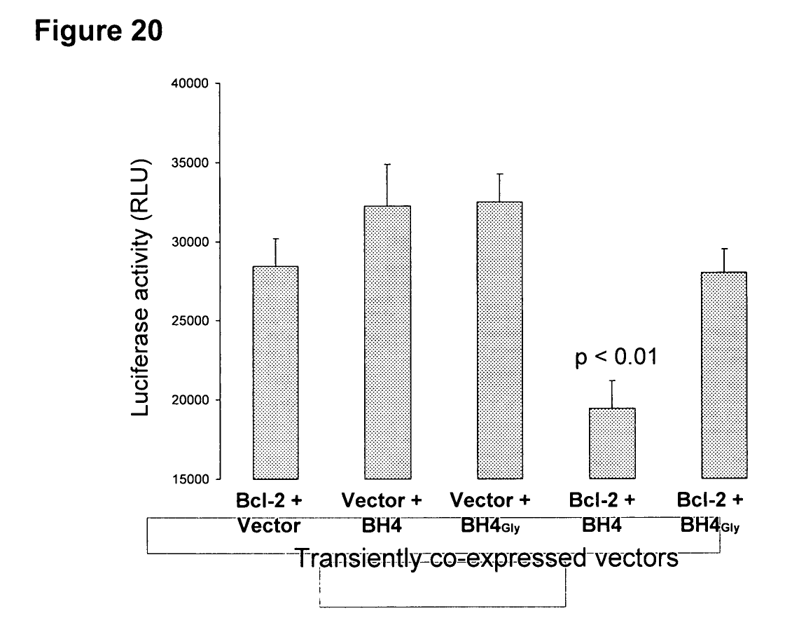

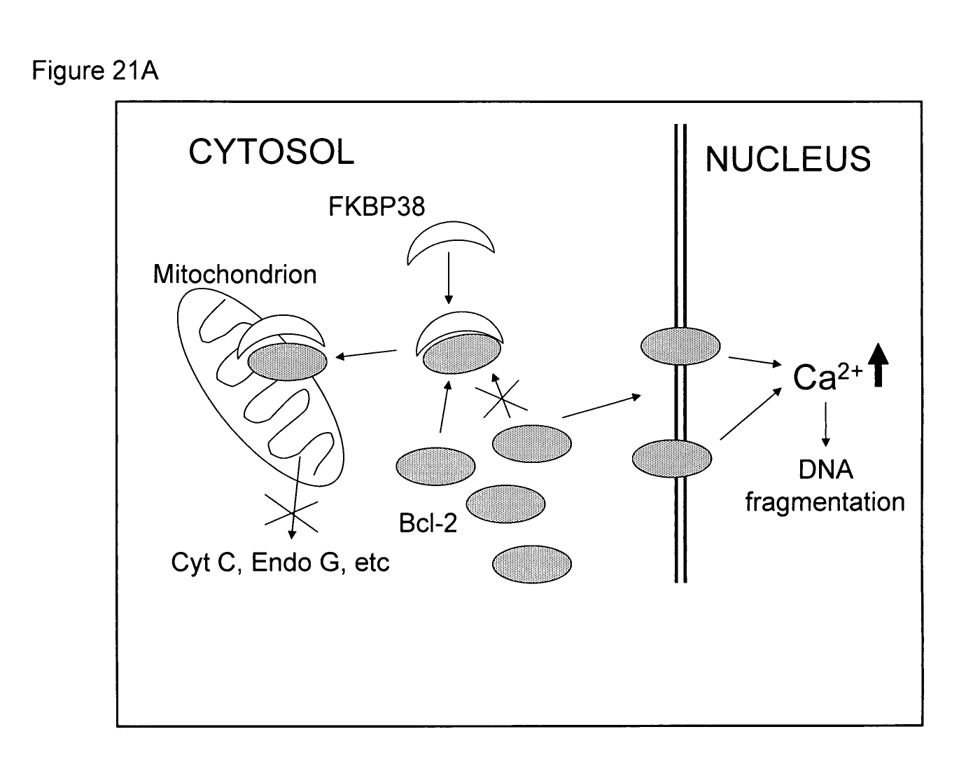

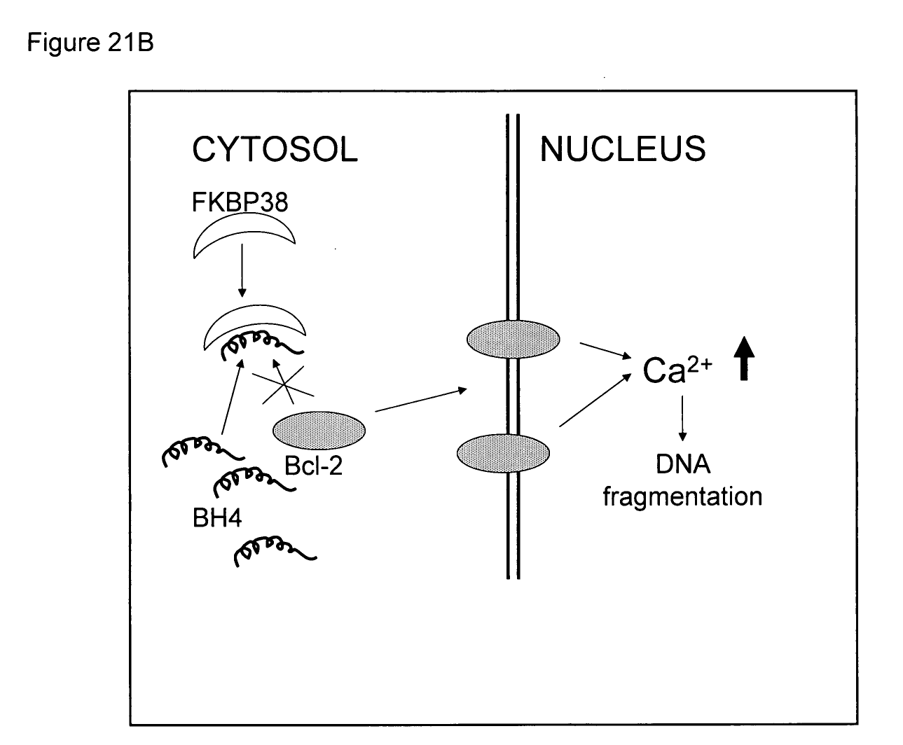

The overall rationale of the experiment was to determine if domain interference disrupted Bcl-2/FKBP38 binding, changed Bcl-2's cellular localization (i.e., result in Bcl-2 misplacement at the nucleus) and ultimately promoted Bcl-2-triggered apoptosis. Preliminary results (FIG. 19) showed that expression of a BH4 peptide induces apoptosis selectively in cells expressing Bcl-2 while leaving cells not expressing Bcl-2 unaffected. In this experiment, cells stably transfected with empty vector (pmKitNeo) or Bcl-2 were further transiently transfected with an expression vector encoding a peptide identical to the last 30 N-terminus amino acids of Bcl-2 including the 11-25 BH4 domain or a mutant inactive BH4 peptide (BH4Gly). The rationale for using a peptide larger that the BH4 domain itself was to provide structure stability to the alpha-helix of the BH4 domain (AA 11-25) through sufficient flanking regions. Cells were further co-transfected with renilla luciferase so as to determine the extent of their survival. Upon transient transfection of the BH4 peptide, the survival of cells stably expressing Bcl-2 was significantly lower as compared to similarly BH4-transfected cells stably expressing the empty pmKitNeo. It is important to note that PC12 cells expressing Bcl-2 cells diddo not depend on Bcl-2 for their survival and therefore the effect of the BH4 peptide was not due to abolishment of Bcl-2 protection. Rather, it reflected an active induction of apoptosis by Bcl-2, triggered by the BH4 peptide. The possibility that PC12 cells expressing Bcl-2 may have undergone selection during the establishment of stable transfection that could have rendered them non-specifically sensitive to the BH4 peptide was excluded. In fact similar experiments in naïve cells transiently (24 hr) co-transfected with Bcl-2 and the BH4 expression vector (FIG. 20) showed that the BH4 peptide alone did not affect cell survival, unless co-transfected with Bcl-2. This result strongly argues against the possibility that the effect of the BH4 peptide shown in FIG. 14 may be non-specific and suggests that the observed cell death is in fact promoted by Bcl-2 in response to the BH4 peptide (FIG. 21B).

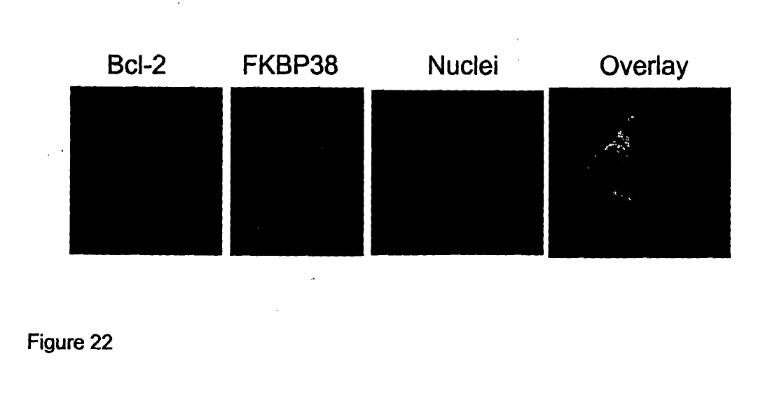

Co-expressed Bcl-2 and FKBP38 co-localize in situ. FIG. 22 shows Bcl-2/FKBP38-cotransfected PC12 cells were stained with appropriate antibodies for Bcl-2 and FKBP38 and for nuclei (using DAPI) and then observed with a confocal microscope. The distribution pattern of Bcl-2 and FKBP38 was very similar and the two, in fact, co-localized. This illustrated that association of Bcl-2 and FKBP38 occurred in situ in living cells and thus excluded that it may have occurred in vitro, after protein isolation.

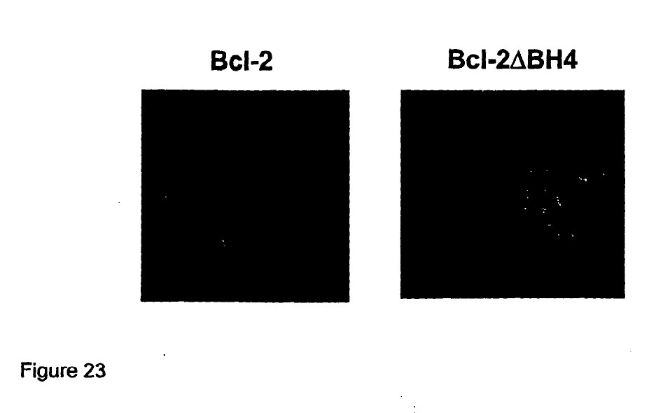

There was a different pattern of sub-cellular distribution of Bcl-2 vs. Bcl-2ΔBH4. FIG. 23 shows Bcl-2 or Bcl-2ΔBH4 transfected PC12 cells that were stained for Bcl-2 and for nuclei (using DAPI) and then observed with a confocal microscope. The distribution of Bcl-2 was cytosolic (consistent with mitochondrial-association) and perinuclear while Bcl-2ΔBH4 was exclusively perinuclear. Thus, the lack of the BH4 domain forced Bcl-2 to the nucleus, consistent with the idea that the BH4 domain is essential to promote Bcl-2/FKBP38 interaction and subsequent proper Bcl-2 distribution to the cytosolic compartment.

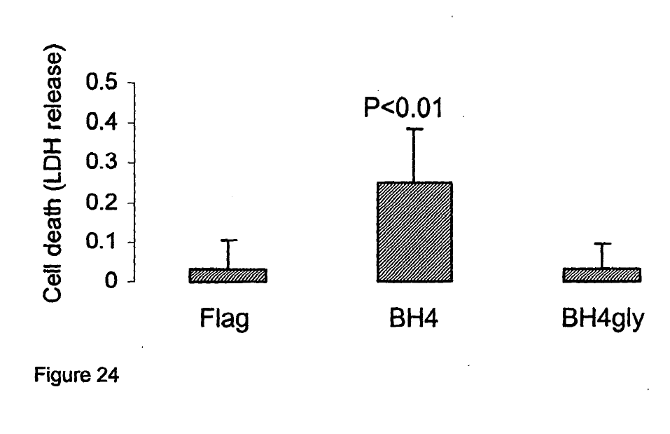

The mutant BH4Gly peptide carries glycine substitutions at amino acids 14 and 15 and is devoid of protein-docking function (Lee L C, et al., (1996)), was ineffective at promoting apoptosis. (FIG. 24) Human leukemia cells (HL-60) were transiently transfected with a BH4 peptide-expressing vector, with the inactive control (BH4Gly) or with empty vector (Flag). The extent of cell death was assessed 48 after transfection as release of LDH in the cell culture medium. Statistical difference was vs. Flag or BH4Gly (Two-tailed Student “t” test; N=4-7 independent measurements per group). This experiment illustrated that interference with FKBP38/Bcl-2 binding by the BH4 peptide was effective in inducing cell death in human leukemia cells, one of the targets of the invention.

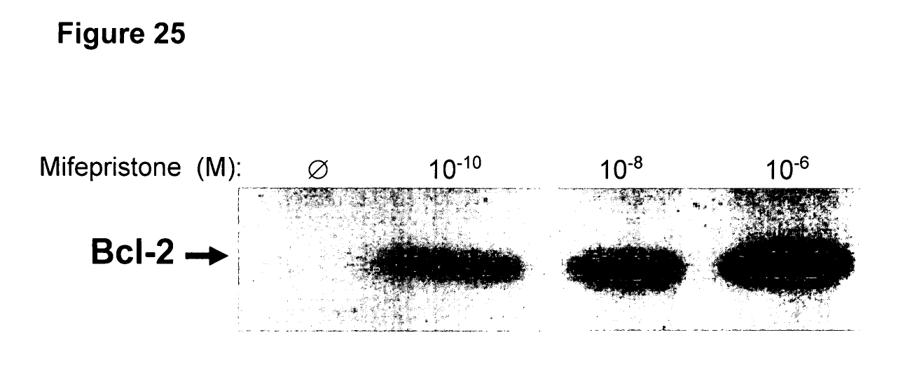

PC12 cells stably transfected with an inducible Bcl-2 expression vector (pGene-Switch) can also be used to assay compounds for their ability to promote apoptosis (FIGS. 25 & 12). The use of an inducible expression system, in addition to producing cells that are stably transfected with Bcl-2 allowed careful modulation of the extent of Bcl-2 expression (FIG. 25). This was necessary to demonstrate that cell sensitivity to BH4-induced apoptosis depended on the extent of Bcl-2 expression, a feature that is important to establish a window of therapy that will specifically target high Bcl-2-expressing leukemia cells and spare normal cells.

### Use of BH4 or Mimetic to Purge Malignant Bcl-2 Producing Cells from a Mixed Population of Cells

Domain interference by a BH4 peptide is a significant novel strategy to overcome drug-resistance and induce apoptosis in leukemia cells. This novel treatment impinges upon apoptosis promoted by Bcl-2, therefore targeting selectively high Bcl-2-expressant AML cells while leaving normal hematopoietic progenitor stem cells, which do not express Bcl-2, unaffected. The molecular mechanisms leading to Bcl-2 nuclear association and induction of apoptosis in response to BH4 7-30 treatment are effective in human myeloid leukemia cell lines, regardless of their sensitivity to conventional chemotherapeutic agents. There exists a causal relationship between sensitivity of leukemia cell lines to BH4 7-30 and the level of Bcl-2 expression, an important feature for specificity of future clinical use. Clinically chemotherapy-sensitive and insensitive malignant cells from AML patients are effectively targeted by the BH4 7-30 peptide while normal CD34+ hematopoietic progenitor cells are not.

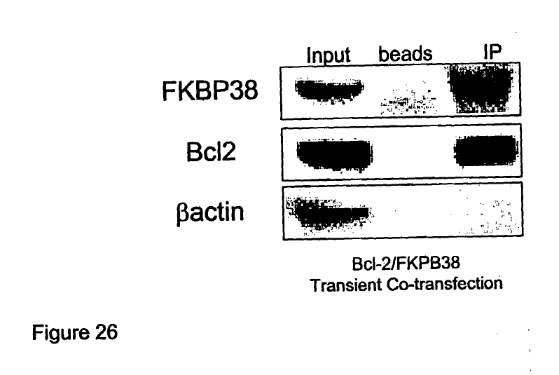

In a separate set of studies employing similarly treated cells, co-immunoprecipitation experiments were performed on total cell protein extracts to determine the extent of the formation of FKBP38/BH4 7-30 complexes. (FIG. 26) In this experiment, PC12 cells were transiently transfected with Bcl-2 in the presence of co-transfected FKBP38. Total cell homogenates were immunoprecipitated with an anti-Bcl-2 antibody to pull down Bcl-2 and analyzed by Western blot probing for FKBP38, Bcl-2 and β-actin. While FKBP38 co-immunoprecipitated with Bcl-2 (indicating interaction between the two proteins), β-actin did not (indicating absence of non-specific protein-protein interactions in the IP reaction). The input lane shows the samples before IP, illustrating proper expression of the protein of interest. The beads used were samples of the agarose beads used for IP, illustrating absence of non-specific binding of the protein of interest to the agarose.

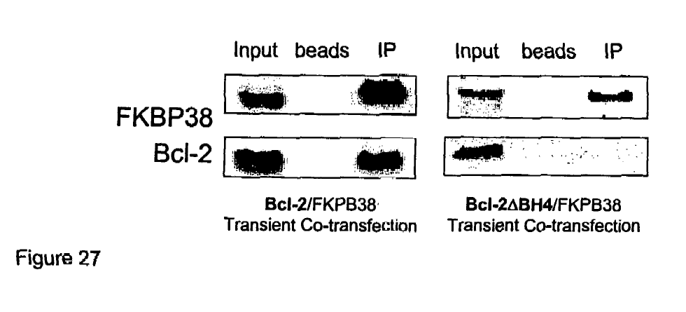

FIG. 27 demonstrates that Bcl-2 lacking the BH4 domain (Bcl-2ΔBH4) did not co-immunoprecipitate with FKBP38 after transient co-transfection in PC12 cells. In this experiment, PC12 cells were transiently transfected with Bcl-2 or Bcl-2ΔBH4 in the presence of co-transfected FKBP38. Total cell homogenates were IP with an anti-HA antibody to pull down FKBP38 (FKBP38 is tagged with HA) and analyzed by Western blot probing for FKBP38 or Bcl-2. Note absence of co-IP'd Bcl-2ΔBH4. The input lane shows the samples before IP, illustrating proper expression of the protein of interest. The beads used were samples of the agarose beads used for IP, illustrating absence of non-specific binding of the protein of interest to the agarose. This experiment illustrates that the presence of the BH4 domain was essential to promote Bcl-2/FKBP binding.

The present invention discloses a method of interfering with Bcl-2 binding to its mitochondrion-targeting carrier protein, FKBP38, thus forcing Bcl-2 promoted DNA fragmentation through increasing intranuclear Ca²⁺. Disrupting Bcl-2/FKBP38 interaction by a competing BH4 peptide caused Bcl-2 to be misplaced at the nucleus and initiate apoptosis, even in the absence of extrinsic apoptotic stimuli. Cells that express a peptide identical to the BH4 domain of Bcl-2 promote apoptosis in Bcl-2-overexpressing cells while leaving cells not expressing Bcl-2 unaffected.

### LITERATURE CITED

Adams J M, Cory S, uang D C—(1998) The conserved N-terminal BH4 domain of Bcl-2 homologues is essential for inhibition of apoptosis and interaction with CED-4. EMBO Journal 17:1029-1039.
Akao Y, Otsuki Y, Kataoka S, Ito Y, Tsujimoto Y (1994) Multiple subcellular localization of bcl-2: detection in nuclear outer membrane, endoplasmic reticulum membrane, and mitochondrial membranes. Cancer Res 54:2468-2471.
Allen R T, Cluck M W, Agrawal D K (1998) Mechanisms controlling cellular suicide: role of Bcl-2 and caspases. Cellular & Molecular Life Sciences 54:427-445.
Andreeff M, Jiang S, Zhang X, Konopleva M, Estrov Z, Snell V E, Xie Z, Okcu M F, Sanchez-Williams G, Dong J, Estey E H, Champlin R C, Kornblau S M, Reed J C, Zhao S (1999) Expression of Bcl-2-related genes in normal and AML progenitors: changes induced by chemotherapy and retinoic acid. Leukemia 13:1881-1892.
Antonsson B (2001) Bax and other pro-apoptotic Bcl-2 family “killer-proteins” and their victim the mitochondrion. Cell & Tissue Research 306:347-361.
Bossy-Wetzel E, Green DR (1999) Apoptosis: checkpoint at the mitochondrial frontier. Mutation Research 434:243-251.
Bouillet P, Strasser A (2002a) BH3-only proteins—evolutionarily conserved proapoptotic Bcl-2 family members essential for initiating programmed cell death. Journal of Cell Science 115:1567-1574.
Bouillet P, Strasser A (2002b) BH3-only proteins—evolutionarily conserved proapoptotic Bcl-2 family members essential for initiating programmed cell death. J Cell Sci 115:1567-1574.
Bradbury D A, Russell N H (1995) Comparative quantitative expression of bcl-2 by normal and leukaemic myeloid cells. Br J Haematol 91:374-379.
Bruckheimer E M, Cho S H, Sarkiss M, Herrmann J, McDonnell T J (1998) The Bcl-2 gene family and apoptosis. Advances in Biochemical Engineering-Biotechnology 62:75-105.
Campos L, Sabido O, Rouault J P, Guyotat D (1994) Effects of BCL-2 antisense oligodeoxynucleotides on in vitro proliferation and survival of normal marrow progenitors and leukemic cells. Blood 84:595-600.
Campos L, Rouault J P, Sabido O, Oriol P, Roubi N, Vasselon C, Archimbaud E, Magaud J P, Guyotat D (1993) High expression of bcl-2 protein in acute myeloid leukemia cells is associated with poor response to chemotherapy. Blood 81:3091-3096.
Carter B Z, Wang R Y, Schober W D, Milella M, Chism D, Andreeff M (2003a) Targeting Survivin expression induces cell proliferation defect and subsequent cell death involving mitochondrial pathway in myeloid leukemic cells. Cell Cycle 2:488-493.
Chandra J, Pataer A, Fang B, Roth J A, Swisher S G, O'Neil R G, McConkey D J, Nutt L K (2002) Bax and Bak promote apoptosis by modulating endoplasmic reticular and mitochondrial Ca2± stores. Journal of Biological Chemistry 277:20301-20308.
Chao D T, Korsmeyer S J (1998) BCL-2 family: regulators of cell death. Annual Review of Immunology 16:395-419.
Cohen-Saidon C, Nechushtan H, Kahlon S, Livni N, Nissim A, Razin E (2003) A novel strategy using single-chain antibody to show the importance of Bcl-2 in mast cell survival. Blood 102:2506-2512.
Cory S, Adams J M (2002) The Bcl2 family: regulators of the cellular life-or-death switch. Nat Rev Cancer 2:647-656.
Cotter F E, Waters J, Cunningham D (1999) Human Bcl-2 antisense therapy for lymphomas. Biochimica et Biophysica Acta 1489:97-106.
De Giorgi F, Lartigue L, Bauer MK, Schubert A, Grimm S, Hanson G T, Remington S J, Youle R J, Ichas F, Grimm D (2002) The permeability transition pore signals apoptosis by directing Bax translocation and multimerization Simulated microgravity alters differentiation and increases apoptosis in human follicular thyroid carcinoma cells. FASEB Journal 16:607-609.
Deng G M, Su J H, Ivins K J, Van Houten B, Cotman C W (1999) Bcl-2 facilitates recovery from DNA damage after oxidative stress. Experimental Neurology 159:309-318.
Distelhorst C W, Shore G C (2004) Bcl-2 and calcium: controversy beneath the surface. Oncogene 23:2875-2880.
Feng W Y, Liu F T, Patwari Y, Agrawal S G, Newland A C, Jia L (2003) BH3-domain mimetic compound BH3I-2′ induces rapid damage to the inner mitochondrial membrane prior to the cytochrome c release from mitochondria. Br J Haematol 121:332-340.
Fisher D E (2001) Pathways of apoptosis and the modulation of cell death in cancer. Hematology—Oncology Clinics of North America 15:931-956.
Frankel S R (2003) Oblimersen sodium (G3139 Bcl-2 antisense oligonucleotide) therapy in Waldenstrom's macroglobulinemia: a targeted approach to enhance apoptosis. Semin Oncol 30:300-304.
Germain M, Shore G C (2003) Cellular distribution of Bcl-2 family proteins. Sci STKE 2003:pe10.
Gleave M E, Miayake H, Goldie J, Nelson C, Tolcher A (1999) Targeting bcl-2 gene to delay androgen-independent progression and enhance chemosensitivity in prostate cancer using antisense bcl-2 oligodeoxynucleotides. Urology 54:36-46.
Goping I S, Gross A, Lavoie J N, Nguyen M, Jemmerson R, Roth K, Korsmeyer S J, Shore G C (1998) Regulated targeting of BAX to mitochondria. Journal of Cell Biology 143:207-215.
Gross A, McDonnell J M, Korsmeyer S J (1999) BCL-2 family members and the mitochondria in apoptosis. Genes & Development 13:1899-1911.
Huang D C, Strasser A (2000) BH3—Only proteins-essential initiators of apoptotic cell death. Cell 103:839-842.
Jiffar T, Kurinna S, Suck G, Carlson-Bremer D, Ricciardi M R, Konopleva M, Andreeff M, Ruvolo P P (2004) PKC alpha mediates chemoresistance in acute lymphoblastic leukemia through effects on Bcl2 phosphorylation. Leukemia 18:505-512.
Juin P, Geneste O, Raimbaud E, Hickman J A (2004) Shooting at survivors: Bcl-2 family members as drug targets for cancer. Biochim Biophys Acta 1644:251-260.
Kaufmann J A, Perez M, Zhang W, Bickford P C, Holmes D B, Taglialatela G (2003) Free radical-dependent nuclear localization of Bcl-2 in the central nervous system of aged rats is not associated with Bcl-2-mediated protection from apoptosis. J Neurochem 87:981-994.
Kirkin V, Joos S, Zornig M (2004) The role of Bcl-2 family members in tumorigenesis. Biochim Biophys Acta 1644:229-249.
Kitada S, Takayama S, De Riel K, Tanaka S, Reed J C (1994) Reversal of chemoresistance of lymphoma cells by antisense-mediated reduction of bcl-2 gene expression. Antisense Res Dev 4:71-79.
Klasa R J, Gillum A M, Klem R E, Frankel S R (2002) Oblimersen bcl-2 antisense: Facilitating apoptosis in anticancer treatment. Antisense & Nucleic Acid Drug Development 12:193-213.
Konopleva M, Konoplev S, Hu W, Zaritskey A Y, Afanasiev B V, Andreeff M (2002a) Stromal cells prevent apoptosis of AML cells by up-regulation of anti-apoptotic proteins. Leukemia 16:1713-1724.
Konopleva M, Tari A M, Estrov Z, Harris D, Xie Z, Zhao S, Lopez-Berestein G, Andreeff M (2000) Liposomal Bcl-2 antisense oligonucleotides enhance proliferation, sensitize acute myeloid leukemia to cytosine-arabinoside, and induce apoptosis independent of other antiapoptotic proteins. Blood 95:3929-3938.
Konopleva M, Zhao S, Xie Z, Segall H, Younes A, Claxton D F, Estrov Z, Komblau S M, Andreeff M (1999) Apoptosis. Molecules and mechanisms. Advances in Experimental Medicine & Biology 457:217-236.
Konopleva M, Zhao S, Hu W, Jiang S, Snell V, Weidner D, Jackson C E, Zhang X, Champlin R, Estey E, Reed J C, Andreeff M (2002b) The anti-apoptotic genes Bcl-X(L) and Bcl-2 are over-expressed and contribute to chemoresistance of non-proliferating leukaemic CD34± cells. Br J Haematol 118:521-534.
Konstantinidou A E, Korkolopoulou P, Patsouris E (2002) Apoptotic markers for tumor recurrence: A minireview. Apoptosis 7:461-470.
Kroemer G (1998) The mitochondrion as an integrator/coordinator of cell death pathways. Cell Death & Differentiation 5:547.
Kroemer G, Reed J C (2000) Mitochondrial control of cell death. Nat Med 6:513-519.
Lee L C, Hunter J J, Mujeeb A, Turck C, Parslow T G (1996) Evidence for alpha-helical conformation of an essential N-terminal region in the human Bcl2 protein. J Biol Chem 271:23284-23288.
Mancini M, Nicholson D W, Roy S, Thornberry N A, Peterson E P, Casciola-Rosen L A, Rosen A (1998) The caspase-3 precursor has a cytosolic and mitochondrial distribution: implications for apoptotic signaling. Journal of Cell Biology 140:1485-1495.
Marani M, Tenev T, Hancock D, Downward J, Lemoine NR (2002) Identification of novel isoforms of the BH3 domain protein Bim which directly activate Bax to trigger apoptosis. Mol Cell Biol 22:3577-3589.
Massaad C A, Taglialatela G (2003) Bcl-2 phosphorylation is not required for its effects on NFkappaB activity. Neuroreport 14:1167-1170.
Maung Z T, MacLean F R, Reid M M, Pearson A D, Proctor S J, Hamilton P J, Hall A G (1994) The relationship between bcl-2 expression and response to chemotherapy in acute leukaemia. Br J Haematol 88:105-109.
Nahta R, Esteva F J (2003) Bcl-2 antisense oligonucleotides: a potential novel strategy for the treatment of breast cancer. Semin Oncol 30:143-149.
Noguchi S, Hirashima N, Furuno T, Nakanishi M (2003) Remarkable induction of apoptosis in cancer cells by a novel cationic liposome complexed with a bcl-2 antisense oligonucleotide. J Control Release 88:313-320.
O'Connor L, Strasser A, O'Reilly L A, Hausmann G, Adams J M, Cory S, Huang D C (1998) Bim: a novel member of the Bcl-2 family that promotes apoptosis. Embo J 17:384-395.
Okuno S, Shimizu S, Ito T, Nomura M, Hamada E, Tsujimoto Y, Matsuda H (1998) Bcl-2 prevents caspaseindependent cell death. Journal of Biological Chemistry 273:34272-34277.
Panaretakis T, Pokrovskaja K, Shoshan M C, Grander D (2002) Activation of Bak, Bax, and BH3-only proteins in the apoptotic response to doxorubicin. J Biol Chem 277:44317-44326.
Pepper C, Thomas A, Hoy T, Cotter F, Bentley P (1999) Antisense-mediated suppression of Bcl-2 highlights its pivotal role in failed apoptosis in B-cell chronic lymphocytic leukaemia. British Journal of Haematology 107:611-615.
Pommier Y, Sordet O, Antony S, Hayward R L, Kohn K W (2004) Apoptosis defects and chemotherapy resistance: molecular interaction maps and networks. Oncogene 23:2934-2949.
Porwit-MacDonald A, Ivory K, Wilkinson S, Wheatley K, Wong L, Janossy G (1995) Bcl-2 protein expression in normal human bone marrow precursors and in acute myelogenous leukemia. Leukemia 9:1191-1198.
Reed J C (1995) Bcl-2: prevention of apoptosis as a mechanism of drug resistance. Hematol Oncol Clin North Am 9:451-473.
Reed J C (1997) Promise and problems of Bcl-2 antisense therapy. J Natl Cancer Inst 89:988-990.
Ruvolo P P, Deng X, Carr B K, May W S (1998) A functional role for mitochondrial protein kinase Calpha in Bcl2 phosphorylation and suppression of apoptosis. Journal of Biological Chemistry 273:25436-25442.
Sato N, Hotta K, Waguri S, Nitatori T, Tohyama K, Tsujimoto Y, Uchiyama Y (1994) Neuronal differentiation of PC12 cells as a result of prevention of cell death by bcl-2. Journal of Neurobiology 25:1227-1234.
Shirane M, Nakayama K I (2003) Inherent calcineurin inhibitor FKBP38 targets Bcl-2 to mitochondria and inhibits apoptosis. Nat Cell Biol 5:28-37.
Song Y S, Park H J, Kim S Y, Lee S H, Yoo H S, Lee H S, Lee M K, Oh K W, Kang S K, Lee S E, Hong J T (2004) Protective role of Bcl-2 on beta-amyloid-induced cell death of differentiated PC12 cells: reduction of NFkappaB and p38 MAP kinase activation. Neurosci Res 49:69-80.
Susin S A, Lorenzo H K, Zamzami N, Marzo I, Snow B E, Brothers G M, Mangion J, Jacotot E, Costantini P, Loeffler M, Larochette N, Goodlett D R, Aebersold R, Siderovski D P, Penninger J M, Kroemer G (1999) Molecular characterization of mitochondrial apoptosis-inducing factor [see comments]. Nature 397:441-446.
Tanabe K, Kim R, Inoue H, Emi M, Uchida Y, Toge T (2003) Antisense Bcl-2 and HER-2 oligonucleotide treatment of breast cancer cells enhances their sensitivity to anticancer drugs. Int J Oncol 22:875-881.
Thomenius M J, Wang N S, Reineks E Z, Wang Z, Distelhorst C W (2003) Bcl-2 on the endoplasmic reticulum regulates Bax activity by binding to BH3-only proteins. J Biol Chem 278:6243-6250.
Tyurina Y Y, Tyurin V A, Carta G, Quinn P J, Schor N F, Kagan V E (1997) Direct evidence for antioxidant effect of Bcl-2 in PC12 rat pheochromocytoma cells. Arch Biochem Biophys 344:413-423.
Uhlmann E J, Subramanian T, Vater C A, Lutz R, Chinnadurai G (1998) A potent cell death activity associated with transient high level expression of BCL-2. J Biol Chem 273:17926-17932.
Wang J L, Liu D, Zhang Z J, Shan S, Han X, Srinivasula S M, Croce C M, Alnemri E S, Huang Z (2000a) Structure based discovery of an organic compound that binds Bcl-2 protein and induces apoptosis of tumor cells. Proceedings of the National Academy of Sciences of the United States of America 97:7124-7129.
Wang J L, Zhang Z J, Choksi S, Shan S, Lu Z, Croce C M, Alnemri E S, Korngold R, Huang Z (2000b) Cell permeable Bcl-2 binding peptides: a chemical approach to apoptosis induction in tumor cells. Cancer Res 60:1498-1502.
Wang N S, Unkila M T, Reineks E Z, Distelhorst C W (2001) Transient expression of wild-type or mitochondrially targeted Bcl-2 induces apoptosis, whereas transient expression of endoplasmic reticulum-targeted Bcl-2 is protective against Bax-induced cell death. J Biol Chem 276:44117-44128.
Wang Z H, Ding M X, Chew-Cheng S B, Yun J P, Chew E C (1999) Bcl-2 and Bax proteins are nuclear matrix associated proteins. Anticancer Res 19:5445-5449.
Waters J S, Webb A, Cunningham D, Clarke P A, Raynaud F, di Stefano F, Cotter F E (2000) Phase I clinical and pharmacokinetic study of bcl-2 antisense oligonucleotide therapy in patients with non-Hodgkin's lymphoma [see comments]. Journal of Clinical Oncology 18:1812-1823.
Wei M C, Zong W X, Cheng E H, Lindsten T, Panoutsakopoulou V, Ross A J, Roth K A, MacGregor G R, Thompson C B, Korsmeyer S J (2001) Proapoptotic BAX and BAK: a requisite gateway to mitochondrial dysfunction and death. Science 292:727-730.
Wolter K G, Hsu Y T, Smith C L, Nechushtan A, Xi X G, Youle R J (1997) Movement of Bax from the cytosol to mitochondria during apoptosis. J Cell Biol 139:1281-1292.
Yamaguchi T, Okada T, Takeuchi K, Tonda T, Ohtaki M, Shinoda S, Masuzawa T, Ozawa K, Inaba T (2003) Enhancement of thymidine kinase-mediated killing of malignant glioma by BimS, a BH3-only cell death activator. Gene Ther 10:375-385.
Yang J, Liu X, Bhalla K, Kim C N, Ibrado A M, Cai J, Peng T I, Jones D P, Wang X (1997) Prevention of apoptosis by Bcl-2: release of cytochrome c from mitochondria blocked. Science 275:1129-1132.
Yuen A R, Sikic B I (2000) Clinical studies of antisense therapy in cancer. Front Biosci 5:D588-593.
Ziegler A, Simoes-Wuest A P, Zangemeister-Wittke U (2000) Optimizing efficacy of antisense oligodeoxynucleotides targeting inhibitors of apoptosis. Methods in Enzymology 314:477-490.
Zong W X, Lindsten T, Ross A J, MacGregor G R, Thompson C B (2001) BH3-only proteins that bind pro-survival Bcl-2 family members fail to induce apoptosis in the absence of Bax and Bak. Genes Dev 15:1481-1486.
Zong W X, Li C, Hatzivassiliou G, Lindsten T, Yu Q C, Yuan J, Thompson C B (2003) Bax and Bak can localize to the endoplasmic reticulum to initiate apoptosis. J Cell Biol 162:59-69.

What is claimed is:

1. A method for screening compounds that disrupt binding between Bcl-2 and FKBP38, said method comprising the steps of:
a. providing a reaction system which assays binding between Bcl-2 and FKBP38;
b. contacting said reaction system with a compound suspected of disrupting binding between Bcl-2 and FKBP38; and
c. measuring the binding between Bcl-2 and FKBP38.

2. The method of claim 1 wherein said binding is BH-4 domain mediated.

3. The method of claim 1 wherein said method is a cell-based assay.

4. The method of claim 1 wherein said method is non-cell based.

5. The method of claim 1 adapted for high-throughput screening.

6. The method of claim 1 further comprising the further step of determining the ability of said compound to trigger Bcl-2 promoted cell death.

7. A method of promoting apoptotic cell death in Bcl-2 producing cells or tissues, the method comprising the step of contacting the cells or tissues with a sufficient amount of a composition comprising BH4 peptide or mimetic thereof to interfere with the binding of Bcl-2 and FKBP38.

8. The method of claim 7 wherein the cells or tissues are cancer cells.

9. The method of claim 8 wherein the cancer cells are in an individual.

10. The method of claim 7 wherein the cells or tissues are selected from the group of cells contributing to malignancies selected from the group consisting of acute lymphoblastic leukemia, precursor B-lymphoblastic leukemia/lymphoma, diffuse large B-cell lymphoma, liposarcoma, squamous cell carcinoma, meningioma, breast carcinoma, gliobastoma, Hodgkin lymphoma, ependymoma, gastrointestinal stromal tumors, leukemia, melanoma, colon cancer, and hormone-refractory breast cancer.

11. The method of claim 7 wherein the cells or tissues are selected from the group of cells contributing to malignancies selected from the group consisting of prostate, colorectal, lung, gastric, renal neuroblastoma, non-Hodgkin's lymphoma, acute leukemia, and chronic leukemia.

12. A method of purging malignant, Bcl-2 producing cells from a mixed population of cells, said method comprising the step of contacting said mixed population with a composition comprising a sufficient amount of BH4 peptide or a mimetic thereof to trigger apoptosis in said Bcl-2 producing cells.

13. The method of claim 12 wherein said cells or tissues are selected from the group of cells contributing to malignancies selected from the group consisting of acute lymphoblastic leukemia, precursor B-lymphoblastic leukemia/lymphoma, diffuse large B-cell lymphoma, liposarcoma, squamous cell carcinoma, meningioma, breast carcinoma, gliobastoma, Hodgkin lymphoma, ependymoma, gastrointestinal stromal tumors, leukemia, melanoma, colon cancer, and hormone-refractory breast cancer.

14. The method of claim 12 wherein the cells or tissues are selected from the group of cells contributing to malignancies selected from the group consisting of prostate, colorectal, lung, gastric, renal neuroblastoma, non-Hodgkin's lymphoma, acute leukemia, and chronic leukemia.

15. The method of claim 11, wherein the mixed population of cells is positioned in or derived from an individual.

16. The method of claim 12 wherein said step of contacting said mixed population with a composition comprising a sufficient amount of BH4 peptide or mimetic thereof cooperates with steps of administering to said mixed population compositions comprising anti-cancer chemotherapeutic compositions.
